(19) United States
(12) Patent Application Publication
(10) Pub. No.: US 2019/0023733 A1
(43) Pub. Date: Jan. 24, 2019

(54) AMINE DERIVATIVES OF LUPANES WITH HIV MATURATION INHIBITORY ACTIVITY

(71) Applicant: GLAXOSMITHKLINE INTELLECTUAL PROPERTY (NO.2) LIMITED, Brentford, Middlesex (GB)

(72) Inventor: Brian Alvin JOHNS, Research Triangle Park, NC (US)

(21) Appl. No.: 16/070,325

(22) PCT Filed: Jan. 19, 2017
(86) PCT No.: PCT/IB2017/050279

§ 371 Date: Jul. 16, 2018

Related U.S. Application Data
(60) Provisional application No. 62/280,809, filed on Jan. 20, 2016.

(51) Int. Cl. C07J 63/00; A61P 31/18

(52) U.S. Cl. CPC C07J 63/008; A61P 31/18

(57) ABSTRACT

The present invention relates to compounds characterized by having a structure according to the following Formula I:

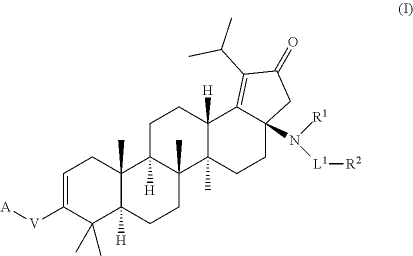

or a pharmaceutically acceptable salt thereof. Compounds of the present invention are useful for the treatment or prevention of HIV.

### CROSS REFERENCE TO RELATED INVENTION

This application claims the benefit of U.S. provisional application Ser. No. 62/280,089 filed Jan. 20, 2016.

### FIELD OF THE INVENTION

The present invention relates to compounds, pharmaceutical compositions, and methods of use thereof for (i) inhibiting HIV replication in a subject infected with HIV, or (ii) treating a subject infected with HIV.

### BACKGROUND OF THE INVENTION

Human immunodeficiency virus type 1 (HIV-1) leads to the contraction of acquired immune deficiency disease (AIDS). The number of cases of HIV continues to rise, and currently over twenty-five million individuals worldwide suffer from the virus. Presently, long-term suppression of viral replication with antiretroviral drugs is the only option for treating HIV-1 infection. Indeed, the U.S. Food and Drug Administration has approved twenty-five drugs over six different inhibitor classes, which have been shown to greatly increase patient survival and quality of life. However, additional therapies are still required because of undesirable drug-drug interactions; drug-food interactions; non-adherence to therapy; and drug resistance due to mutation of the enzyme target.

Currently, almost all HIV positive patients are treated with therapeutic regimens of antiretroviral drug combinations termed, highly active antiretroviral therapy (“HAART”). However, HAART therapies are often complex because a combination of different drugs must be administered often daily to the patient to avoid the rapid emergence of drug-resistant HIV-1 variants. Despite the positive impact of HAART on patient survival, drug resistance can still occur. The emergence of multidrug-resistant HIV-1 isolates has serious clinical consequences and must be suppressed with a new drug regimen, known as salvage therapy.

Current guidelines recommend that salvage therapy includes at least two, and preferably three, fully active drugs. Typically, first-line therapies combine three to four drugs targeting the viral enzymes reverse transcriptase and protease. One option for salvage therapy is to administer different combinations of drugs from the same mechanistic class that remain active against the resistant isolates. However, the options for this approach are often limited, as resistant mutations frequently confer broad cross-resistance to different drugs in the same class. Alternative therapeutic strategies have recently become available with the development of fusion, entry, and integrase inhibitors. However, resistance to all three new drug classes has already been reported both in the lab and in patients. Sustained successful treatment of HIV-1-infected patients with antiretroviral drugs will therefore require the continued development of new and improved drugs with new targets and mechanisms of action.

Presently, long-term suppression of viral replication with antiretroviral drugs is the only option for treating HIV-1 infection. To date, a number of approved drugs have been shown to greatly increase patient survival. However, therapeutic regimens known as highly active antiretroviral therapy (HAART) are often complex because a combination of different drugs must be administered to the patient to avoid the rapid emergence of drug-resistant HIV-1 variants. Despite the positive impact of HAART on patient survival, drug resistance can still occur.

The HIV Gag polyprotein precursor (Pr55Gag), which is composed of four protein domains—matrix (MA), capsid (CA), nucleocapsid (NC) and p6—and two spacer peptides, SP1 and SP2, represents a new therapeutic target. Although the cleavage of the Gag polyprotein plays a central role in the progression of infectious virus particle production, to date, no antiretroviral drug has been approved for this mechanism.

In most cell types, assembly occurs at the plasma membrane, and the MA domain of Gag mediates membrane binding. Assembly is completed by budding of the immature particle from the cell. Concomitant with particle release, the virally encoded PR cleaves Gag into the four mature protein domains, MA, CA, NC and p6, and the two spacer peptides, SP1 and SP2. Gag-Pol is also cleaved by PR, liberating the viral enzymes PR, RT and IN. Gag proteolytic processing induces a morphological rearrangement within the particle, known as maturation. Maturation converts the immature, donut-shaped particle to the mature virion, which contains a condensed conical core composed of a CA shell surrounding the viral RNA genome in a complex with NC and the viral enzymes RT and IN. Maturation prepares the virus for infection of a new cell and is absolutely essential for particle infectivity.

Bevirimat (PA-457) is a maturation inhibitor that inhibits the final step in the processing of Gag, the conversion of capsid-SP1 (p25) to capsid, which is required for the formation of infectious viral particles. Bevirimat has activity against ART-resistant and wild-type HIV, and has shown synergy with antiretrovirals from all classes. Bevirimat reduced HIV viral load by a mean of 1.3 log₁₀/mL in patients who achieved trough levels of >=20 μg/mL and who did not have any of the key baseline Gag polymorphisms at Q369, V370 or T371. However, Bevirimat users with Gag polymorphisms at Q369, V370 or T371 demonstrated significantly lower load reductions than patients without Gag polymorphisms at these sites.

Other examples of maturation inhibitors can be found in PCT Patent Application No. WO2011/100308, PCT Patent Application No. PCT/US2012/024288, Chinese PCT Application No. PCT/CN2011/001302, Chinese PCT Application No. PCT/CN2011/001303, Chinese PCT Application No. PCT/CN2011/002105, PCT/CN2011/002159, WO2013/090664, WO2013/123019, WO 2013/043778, WO 2014/123889, WO 2011/153315, WO 2011/153319, WO 2012/106188, WO 2012/106190, WO 2013/169578, WO 2014/13081. Maturation inhibitors in the prior art leave open gaps in the areas of polymorphism coverage whereby potency against a broad range of clinically relevant gag sequences is extremely important, along with overall potency including the clinically relevant protein adjusted antiviral activity that will be required for robust efficacy in long term durability trials. To date, no maturation inhibitor has achieved an optimal balance of these properties.

It would therefore be an advance in the art to discover alternative compounds that are an effective balance of the aforementioned properties for the prevention and/or treatment of HIV infections.

### SUMMARY OF THE INVENTION

Briefly, in one aspect, the present invention provides compounds of Formula I:

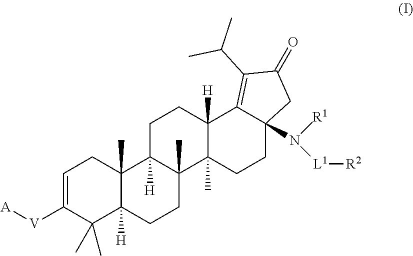

or a pharmaceutically acceptable salt thereof, wherein:

R¹ is H or C₁₋₄alkyl;

L¹ is a bond, C₁₋₈alkylene, or C₀₋₃alkyleneC(O)C₀₋₃alkylene;

R² is N(R³)R⁴, or —OR³, wherein each R³ and R⁴ are independently H, C₁₋₃alkyl, C₁₋₃alkylene-C₃₋₆cycloalkyl, C₁₋₃alkylene-C₅₋₇heterocycle, C₁₋₃alkyleneN(C₁₋₃alkyl)₂, C₁₋₃alkyleneC₅₋₇aryl, C(O)C₅₋₇aryl, C(O)C₁₋₄alkyl, C₁₋₃alkyleneC₅₋₇heteroaryl wherein each R³ and R⁴ may be substituted with one or two substituents independently selected from halogen, C₁₋₄alkyl, SO₂C₁₋₃alkyl; or when R² is N(R³)R⁴, R³ and R⁴ may bond together and along with the N to which they are bonded form a 4 to 7-membered heterocycle, and said heterocycle may contain one other hetero atom selected from N, S, and O and wherein said S atom may be substituted by up to 2 oxo groups, and wherein said heterocycle may be substituted with one or two substituents independently selected from halogen, C₁₋₄alkyl, SO₂C₁₋₃alkyl;

V is C₄₋₈cycloalkylene, C₄₋₈cycloalkenylene, C₅₋₈aryl, or 5 to 8-membered heteroaryl ring, wherein V may be substituted with one or two substituents independently selected from halogen and C₁₋₃alkyl wherein said alkyl may be substituted with one or 2 halogens; and

A is —CO₂C₁₋₃alkyl or CO₂H.

In another aspect, the present invention provides pharmaceutical compositions comprising a compound or salt of the invention.

In another aspect, the present invention provides a method of (i) inhibiting HIV replication in a subject infected with HIV, (ii) treating a subject infected with HIV, or (iii) preventing HIV invention in a subject at risk of such infection.

In another aspect, the present invention provides a compound or salt of the invention for use in therapy, and in particular, in human medicine.

In another aspect, the present invention provides the use of a compound of the invention for the manufacture of a medicament for the (i) inhibiting HIV replication in a subject infected with HIV, (ii) treating a subject infected with HIV, or (iii) preventing HIV invention in a subject at risk of such infection.

### DETAILED DESCRIPTION OF REPRESENTATIVE EMBODIMENTS

Preferably R¹ is H.

Preferably L¹ is C₁₋₄alkylene, C(O), or C(O)C₁₋₃alkylene.

Preferably R² is N(R³)(R⁴).

Preferably R³ and R⁴ are independently C₁₋₃alkyleneN(C₁₋₃alkyl)₂, or C₁₋₃alkylenephenyl, wherein each R³ and R⁴ may be substituted with one substitutent independently selected from halogen or SO₂C₁₋₃alkyl; or R³ and R⁴ are bonded together and along with the N to which they are bonded form a 6-membered heterocycle, and said heterocycle may contain one S atom and wherein said S atom may be substituted by 2 oxo groups; and wherein each R³ and R⁴ may be substituted with one or two substituents independently selected from halogen, C₁₋₄alkyl, SO₂C₁₋₃alkyl.

Preferably V is phenylene or cyclohexenylene optionally substituted with halogen or C₁₋₃alkyl wherein said alkyl may be substituted with one or 2 halogens.

Preferably A is —CO₂H.

Preferably, in the methods of this invention the subject is a human.

Pharmaceutically acceptable salts may be derived from a variety of organic and inorganic counter ions well known in the art and include, by way of example only, sodium, potassium, calcium, magnesium, ammonium, and tetraalkylammonium, and when the molecule contains a basic functionality, salts of organic or inorganic acids, such as hydrochloride, hydrobromide, tartrate, mesylate, acetate, maleate, lysine, and oxalate. Suitable salts include those described in P. Heinrich Stahl, Camille G. Wermuth (Eds.), Handbook of Pharmaceutical Salts Properties, Selection, and Use; 2002.

The present invention may be used in combination with one or more agents useful as pharmacological enhancers as well as with or without additional compounds for the prevention or treatment of HIV. Examples of such pharmacological enhancers (or pharmakinetic boosters) include, but are not limited to, ritonavir and Cobicistat (formerly GS-9350).

### EXAMPLES

The following examples serve to more fully describe the manner of making and using the above-described invention. It is understood that these examples in no way serve to limit the true scope of the invention, but rather are presented for illustrative purposes. In the examples below and the synthetic schemes above, the following abbreviations have the following meanings. If an abbreviation is not defined, it has its generally accepted meaning.

aq.=aqueous

μL=microliters

μM=micromolar

NMR=nuclear magnetic resonance

boc=tert-butoxycarbonyl

br=broad

Cbz=benzyloxycarbonyl

d=doublet

b=chemical shift

OC=degrees celcius

DCE=1,2-dichloroethene

DCM=dichloromethane

dd=doublet of doublets

DIEA or DIPEA=N,N-diisopropylethylamine

DMEM=Dulbeco's Modified Eagle's Medium

DMF=N,N-dimethylformamide

DMP=Dess-Martin periodinane

DMSO=dimethylsulfoxide

DPPA=diphenoxyphosphoryl azide

FA=formic acid

EtOAc=ethyl acetate

g=gram

h or hr=hours

HBTU=2-(1H-benzotriazol-1-yl)-1,1,3,3-tetramethyluronium hexafluorophosphate

HCV=hepatitus C virus

HPLC=high performance liquid chromatography

Hz=hertz

IU=International Units

IC₅₀=inhibitory concentration at 50% inhibition

J=coupling constant (given in Hz unless

otherwise indicated)

K-HMDS=potassium bis(trimethylsilyl)amide

m=multiplet

M=molar

M+^{H}=parent mass spectrum peak plus H

mg=milligram

min=minutes

mL=milliliter

mM=millimolar

mmol=millimole

MS=mass spectrum

N=normal

nm=nanomolar

PE=petroleum ether

PCC=Pyridinium chlorochromate

ppm=parts per million

q.s.=sufficient amount

s=singlet

RT=room temperature

sat.=saturated

t=triplet

TBAF=tetra-n-butylammonium fluoride

TBSCI=tert-butyldimethylsilyl chloride

TEA=triethylamine

tetrakis=tetrakis(triphenylphosphine)palladium(0)

TFA=trifluoroacetic acid

THF=tetrahydrofuran

UPLC=ultra performance liquid chromatography

### Equipment Description

¹H NMR spectra were recorded on a Bruker Ascend 400 spectrometer. Chemical shifts are expressed in parts per million (ppm, 8 units). Coupling constants are in units of hertz (Hz). Splitting patterns describe apparent multiplicities and are designated as s (singlet), d (doublet), t (triplet), q (quartet), quint (quintet), m (multiplet), br (broad).

The analytical low-resolution mass spectra (MS) were recorded on Waters ACQUITY UPLC with SQ Detector using a Waters BEH C18, 2.1×50 mm, 1.7 μm using a gradient elution method.

Solvent A: 0.1% formic acid (FA) in water;

Solvent B: 0.1% FA in acetonitrile;

30% B for 0.5 min followed by 30%-100% B over 2.5 min.

### Schemes and Experimental Procedures

The following schemes and procedures illustrate how compounds of the present invention can be prepared. The specific solvents and reaction conditions referred to are also illustrative and are not intended to be limiting. Compounds not described are either commercially available or are readily prepared by one skilled in the art using available starting materials. The Examples disclosed herein are for illustrative purposes only and are not intended to limit the scope of the invention. All examples exhibited LHIV IC₅₀ values between 21 μM and 1 nM using the assay disclosed herein.

For several of the examples the stereochemistry of the C28 secondary alcohol when present was not definitively confirmed as to its absolute configuration. Unless stated otherwise, the compounds exemplified in the present application were isolated as optically pure stereoisomers and initially assigned to a configuration as drawn. There is the possibility that some of these may be listed as the opposite stereochemistry at that single C28 position as shown. This in no way is meant to limit the scope of the invention or utility of the compounds of Formula I. Additional examples contained within were determined to have the shown configuration by spectroscopic methods well known to those skilled in the art including, but not limited to, 1D and 2D NMR methods, vibrational circular dichroism and X-ray crystallography. These examples and the methods to make both diastereomers should serve to clearly exemplify the pure stereoisomers of both R and S configuration at the C28 position are readily obtained, separated and characterized and any remaining undefined examples could be readily confirmed by similar methods well known to one skilled in the art.

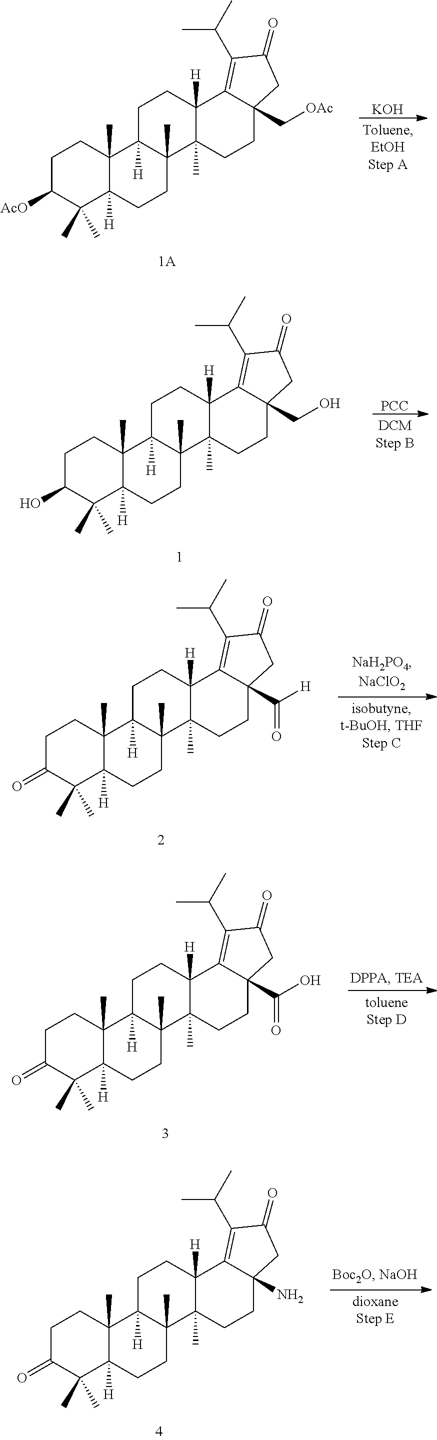

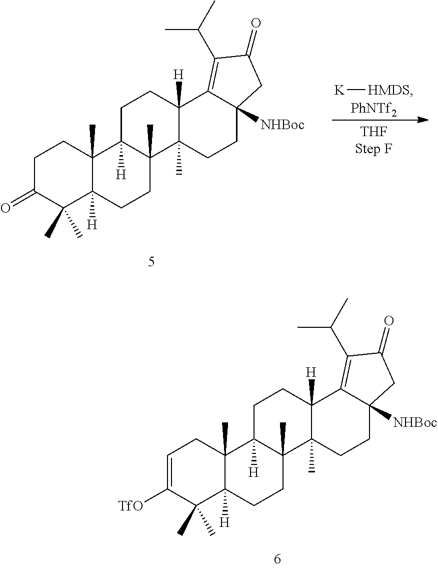

### Step A: Intermediate 1: (3aR,5aR,5bR,7aR,9S,11aR,11bR,13aS)-9-Hydroxy-3a-(hydroxymethyl)-1-isopropyl-5a,5b,8,8,11a-pentamethyl-3,3a,4,5,5a,5b,6,7,7a,8,9,10,11,11a,11b,12,13,13a-octadecahydro-2H-cyclopenta[a]chrysene-2-one

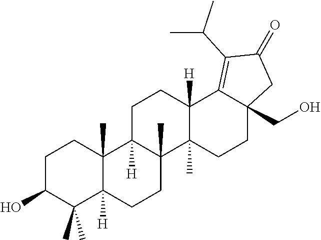

A mixture of intermediate 1A, WO2013/090664, (40 g, 74 mmol) and KOH (16.6 g, 296 mmol) in EtOH (200 mL) and toluene (200 mL) was stirred at room temperature overnight. The resulting mixture was neutralized with 6N HCl and concentrated reduced pressure to remove the volatiles. The residue was partitioned between DCM and H₂O and the layers were separated. The organic layer was washed with brine, dried over Na₂SO₄, filtered and concentrated under reduced pressure to give intermediate 1 (27.4 g, 81% yield) which was directly used in the next step without further purification. LC/MS: m/z calculated 456.4, found 457.5 (M+1)+.

### Step B: Intermediate 2: (3aR,5aR,5bR,7aR,11aR,11bR,13aS)-1-Isopropyl-5a,5b,8,8,11a-pentamethyl-2,9-dioxo-3,3a,4,5,5a,5b,6,7,7a,8,9,10,11,11a,11b,12,13,13a-octadecahydro-2H-cyclopenta[a]chrysene-3a-carbaldehyde

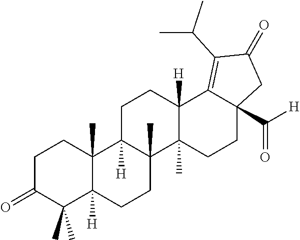

A mixture of intermediate 1 (1 g, 2.2 mmol) and PCC (940 mg, 4.4 mmol) in DCM (20 mL) was stirred at room temperature overnight. The resulting mixture was diluted with DCM and filtered through a pad of Celite. The filtrate was concentrated under reduced pressure to give a product which was purified by flash chromatography (silica gel, 0-30% EtOAc in PE) to afford intermediate 2 (398 mg, 40% yield) as a white solid. LC/MS: m/z calculated 452.3, found 453.5 (M+1)+.

### Step C: Intermediate 3: (3aR,5aR,5bR,7aR,11aR,11bR,13aS)-1-Isopropyl-5a,5b,8,8,11a-pentamethyl-2,9-dioxo-3,3a,4,5,5a,5b,6,7,7a,8,9,10,11,11a,11b,12,13,13a-octadecahydro-2H-cyclopenta[a]chrysene-3a-carboxylic acid

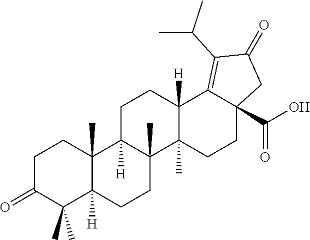

A mixture of intermediate 2 (3 g, 6.6 mmol), NaH₂PO₄ (4.8 g, 40 mmol), NaClO₂ (3.6 g, 40 mmol) in t-BuOH (20 mL), H₂O (30 mL), and THF (25 mL) was treated with isobutyne (15 mL). After stirred at room temperature for 2 hr, the resulting mixture was diluted with H₂O and extracted with EtOAc. The organic layer was washed with sat. Na₂S₂O₃ and brine, dried over Na₂SO₄, filtered and concentrated under reduced pressure to give the crude product which was purified by flash chromatography (silica gel, 0-50% EtOAc in PE) to afford intermediate 3 (2.3 g, 74% yield) as a white solid. ¹H NMR (400 MHz, CDCl₃) δ 10.37 (br, 1H), 3.27-3.15 (m, 1H), 2.79 (dd, J=12.7, 3.0 Hz, 1H), 2.66-2.41 (m, 4H), 2.22 (d, J=18.7 Hz, 1H), 2.09-1.86 (m, 4H), 1.65-1.21 (m, 18H), 1.11-0.96 (m, 14H). LC/MS: m/z calculated 468.3, found 469.4 (M+1)+.

### Step D: Intermediate 4: (3aR,5aR,5bR,7aR,11aR,11bR,13aS)-3a-Amino-1-isopropyl-5a,5b,8,8,11a-pentamethyl-3a,4,5,5a,5b,6,7,7a,8,10,11,11a,11b,12,13,13a-hexadecahydro-2H-cyclopenta[a]chrysene-2,9(3H)-dione

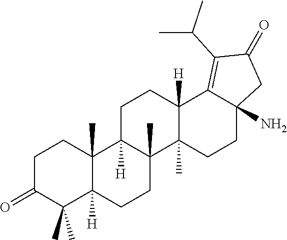

To a solution of intermediate 3 (600 mg, 1.28 mmol) and TEA (0.21 mL, 1.53 mmol) in toluene (6 mL) was added DPPA (422 mg, 1.53 mmol). The reaction was stirred at room temperature for 30 min, and was then concentrated under reduced pressure. The residue was partitioned between EtOAc and H₂O. The organic layer was washed with brine, dried over Na₂SO₄, filtered and concentrated under reduced pressure to give the crude acyl azide intermediate which was dissolved in toluene (10 mL) and heated at 80° C. for 30 min to give the isocyante. The resulting mixture was concentrated and the residue was treated with conc. HCl (5 mL) in DCM (5 mL). The mixture was stirred overnight at room temperature and was concentrated under reduced pressure to give a residue that was triturated in EtOH to afford the intermediate 4 (278 mg, 49% yield) as a white solid after filtration. LC/MS: m/z calculated 439.4, found 440.3 (M+1)+.

### Step E: Intermediate 5: tert-Butyl ((3aR,5aR,5bR,7aR,11aR,11bR,13aS)-1-isopropyl-5a,5b,8,8,11a-pentamethyl-2,9-dioxo-3,3a,4,5,5a,5b,6,7,7a,8,9,10,11,11a,11b,12,13,13a-octadecahydro-2H-cyclopenta[a]chrysen-3a-yl)carbamate

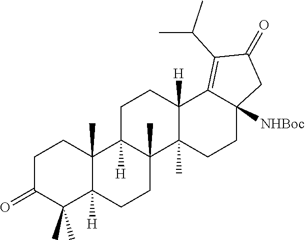

To a solution of intermediate 4 (2 g, 4.5 mmol) in dioxane (20 mL) and 1N NaOH (9 mL) was added Boc₂O (1.7 g, 7.7 mmol). The reaction was stirred at room temperature overnight, then was neutralized with 1N HCl and extracted with EtOAc. The organic layer was washed with brine, dried over Na₂SO₄, filtered and concentrated under reduced pressure to give a residue which was purified by silica gel chromatography (0-50% EtOAc/PE) to afford intermediate 5 (1.9 g, 77% yield) as a white solid. LC/MS: m/z calculated 539.4, found 540.4 (M+1)+.

### Step F: Intermediate 6: (3aR,5aR,5bR,7aR,11aR,11bR,13aS)-3a-((tert-Butoxycarbonyl)amino)-1-isopropyl-5a,5b,8,8,11a-pentamethyl-2-oxo-3,3a,4,5,5a,5b,6,7,7a,8,11,11a,11b,12,13,13a-hexadecahydro-2H-cyclopenta[a]chrysen-9-yl trifluoromethanesulfonate

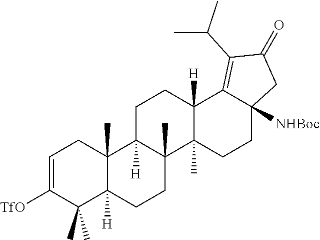

At −78° C., to a solution of intermediate 5 (1.6 g, 3.0 mmol) in anhydrous THF (16 mL) was added K-HMDS (1M, 13.5 mL, 13.5 mmol) dropwise under N₂ atmosphere. The reaction was stirred at −78° C. for 2 hr, then a solution of PhNTf₂ (1.3 g, 3.7 mmol) in anhydrous THF (8 mL) was added dropwise. The reaction was stirred at −78° C. for another 2 hr and slowly warmed up to room temperature. The resulting mixture was quenched with sat. NH₄Cl aq. solution and extracted with EtOAc. The organic layer was washed with brine, dried over Na₂SO₄, filtered and concentrated under reduced pressure to give a residue that was purified by flash chromatography (silica gel, 0-15% EtOAc in PE) to afford intermediate 6 (1.5 g, 75% yield) as a white solid. LC/MS: m/z calculated 671.4, found 672.3 (M+1)+. ¹H NMR (400 MHz, CDCl₃) b 5.59 (dd, J=6.7, 1.9 Hz, 1H), 4.60 (s, 1H), 3.12 (dt, J=13.9, 7.0 Hz, 1H), 2.90 (d, J=11.9 Hz, 1H), 2.66 (d, J=18.6 Hz, 1H), 2.37-2.20 (m, 3H), 2.06-1.80 (m, 5H), 1.58-1.40 (m, 17H), 1.29-1.13 (m, 14H), 1.05-0.91 (m, 8H).

### Synthesis of Intermediate 8

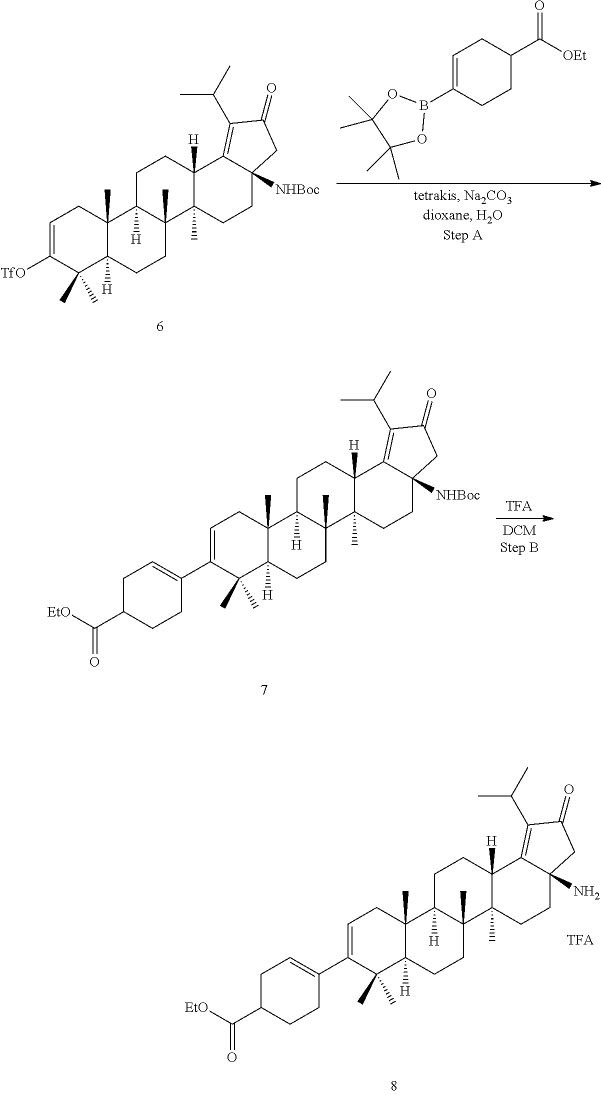

### Step A: Intermediate 7: Ethyl 4-((3aR,5aR,5bR,7aR,11aS,11bR,13aS)-3a-((tert-butoxycarbonyl)amino)-1-isopropyl-5a,5b,8,8,11a-pentamethyl-2-oxo-3,3a,4,5,5a,5b,6,7,7a,8,11,11a,11b,12,13,13a-hexadecahydro-2H-cyclopenta[a]chrysen-9-yl)cyclohex-3-enecarboxylate

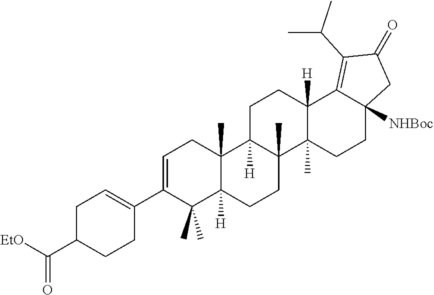

A mixture of intermediate 6 (50 mg, 0.07 mmol), ethyl 4-(4,4,5,5-tetramethyl-1,3,2-dioxaborolan-2-yl)cyclohex-3-ene-1-carboxylate (22 mg, 0.08 mmol), tetrakis (5 mg, 0.0037 mmol) and Na₂CO₃ (24 mg, 0.22 mmol) in dioxane (2 mL) and H₂O (0.5 mL) was purged with N₂ three times. The mixture was heated at 85° C. for 4 hr, the resulting mixture was filtered through a pad of Celite and the filtrate was partitioned between EtOAc and H₂O. The organic layer was washed with brine, dried over Na₂SO₄, filtered and concentrated under reduced pressure to give a residue that was purified by silica gel chromatography (0-15% EtOAc/PE) to afford intermediate 7 (45 mg, 89% yield) as a white solid. LC/MS: m/z calculated 675.5, found 676.4 (M+1)+.

### Step B: Intermediate 8: Ethyl 4-((3aR,5aR,5bR,7aR,11aS,11bR,13aS)-3a-amino-1-isopropyl-5a,5b,8,8,11a-pentamethyl-2-oxo-3,3a,4,5,5a,5b,6,7,7a,8,11,11a,11b,12,13,13a-hexadecahydro-2H-cyclopenta[a]chrysen-9-yl)cyclohex-3-enecarboxylate trifluoroacetic acid salt

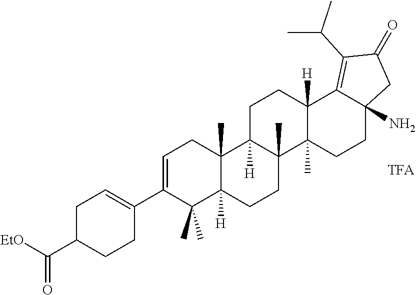

A solution of intermediate 7 (70 mg, 0.10 mmol) in TFA (2 mL) and DCM (2 mL) was stirred at room temperature for 2.5 hr. The resulting mixture was concentrated under reduced pressure to give the intermediate 8 (quant. yield) as a white solid which was used in the next step without purification. LC/MS: m/z calculated 575.4, found 576.4 (M+1)+.

### Example 1 (Compound 10): 4-((3aR,5aR,5bR,7aR,11aS,11bR,13aS)-1-Isopropyl-5a,5b,8,8,11a-pentamethyl-3a-((2-(4-(methylsulfonyl)piperidin-1-yl)ethyl)amino)-2-oxo-3,3a,4,5,5a,5b,6,7,7a,8,11,11a,11b,12,13,13a-hexadecahydro-2H-cyclopenta[a]chrysen-9-yl)cyclohex-3-enecarboxylic acid

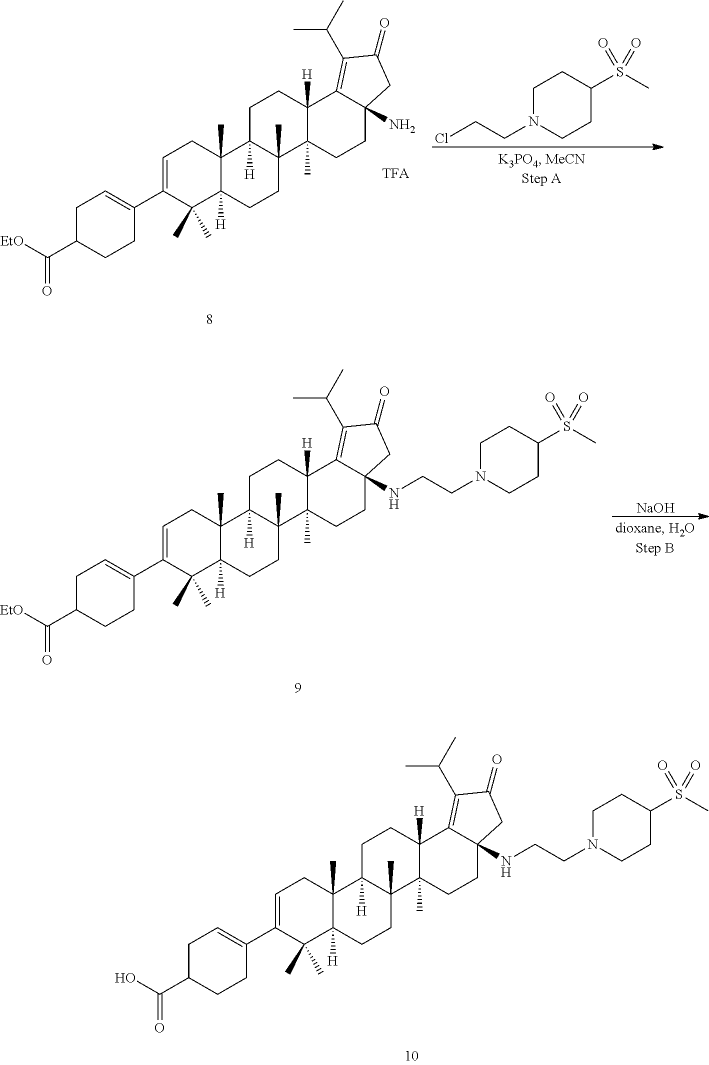

### Step A: Intermediate 9: Ethyl 4-((3aR,5aR,5bR,7aR,11aS,11bR,13aS)-1-isopropyl-5a,5b,8,8,11a-pentamethyl-3a-((2-(4-(methylsulfonyl)piperidin-1-yl)ethyl)amino)-2-oxo-3,3a, 4,5,5a,5b,6,7,7a,8,11,11a,11b,12,13,13a-hexadecahydro-2H-cyclopenta[a]chrysen-9-yl)cyclohex-3-enecarboxylate

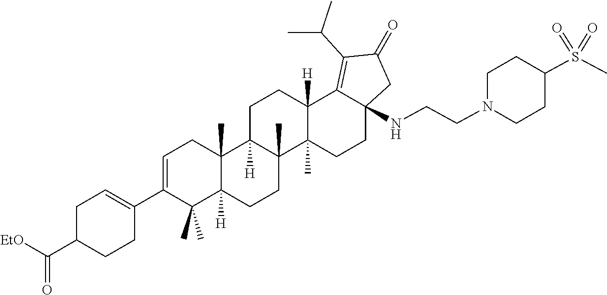

A mixture of intermediate 8 (35 mg, 0.06 mmol), 1-(2-chloroethyl)-4-(methylsulfonyl) piperidine (55 mg, 0.12 mmol) and K₃PO₄ (64 mg, 0.3 mmol) in anhydrous MeCN (1.0 mL) was heated up to 100° C. and stirred overnight under N₂ atmosphere. The resulting mixture was partitioned between EtOAc and H₂O. The organic layer was washed with brine, dried over Na₂SO₄, filtered and concentrated under reduced pressure to give a residue which was purified by silica gel chromatography (0-5% MeOH/DCM) to afford intermediate 9 (16 mg, 35% yield) as a yellow oil. LC/MS: m/z calculated 764.5, found 765.5 (M+1)+.

### Step B: Compound 10 (Example 1: 4-((3aR,5aR,5bR,7aR,11aS,11bR,13aS)-1-Isopropyl-5a,5b,8,8,11a-pentamethyl-3a-((2-(4-(methylsulfonyl)piperidin-1-yl)ethyl)amino)-2-oxo-3,3a,4,5,5a,5b,6,7,7a,8,11,11a,11b,12,13,13a-hexadecahydro-2H-cyclopenta[a]chrysen-9-yl)cyclohex-3-enecarboxylic acid

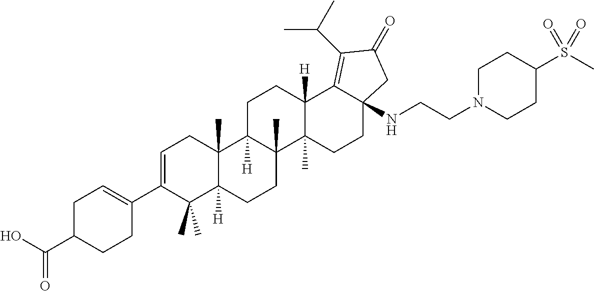

A mixture of intermediate 9 (16 mg, 0.02 mmol) in dioxane (1 mL) and 1N NaOH (0.1 mL) was stirred at 75° C. overnight. The reaction was cooled then diluted with EtOAc and acidified with 1N HCl to pH=3-4. The layers were separated and the organic layer was washed with brine, dried over Na₂SO₄, filtered and concentrated under reduced pressure to give a residue which was purified by reverse phase chromatography (5-100% MeCN/H₂O+0.1% FA) to afford compound 10 (4.4 mg, 30% yield). LC/MS: m/z calculated 736.5, found 737.4 (M+1)+. ¹H NMR (400 MHz, DMSO) b 8.23 (s, 1H), 5.50-5.16 (m, J=91.8, 40.0 Hz, 2H), 3.23-2.76 (m, 8H), 2.50-0.71 (m, 57H).

### Example 2 (Compound 12): 4-((3aR,5aR,5bR,7aR,11aS,11bR,13aS)-3a-((2-(1,1-Dioxidothiomorpholino)ethyl)amino)-1-isopropyl-5a,5b,8,8,11a-pentamethyl-2-oxo-3,3a,4,5,5a,5b,6,7,7a,8,11,11a,11b,12,13,13a-hexadecahydro-2H-cyclopenta[a]chrysen-9-yl)cyclohex-3-enecarboxylic acid

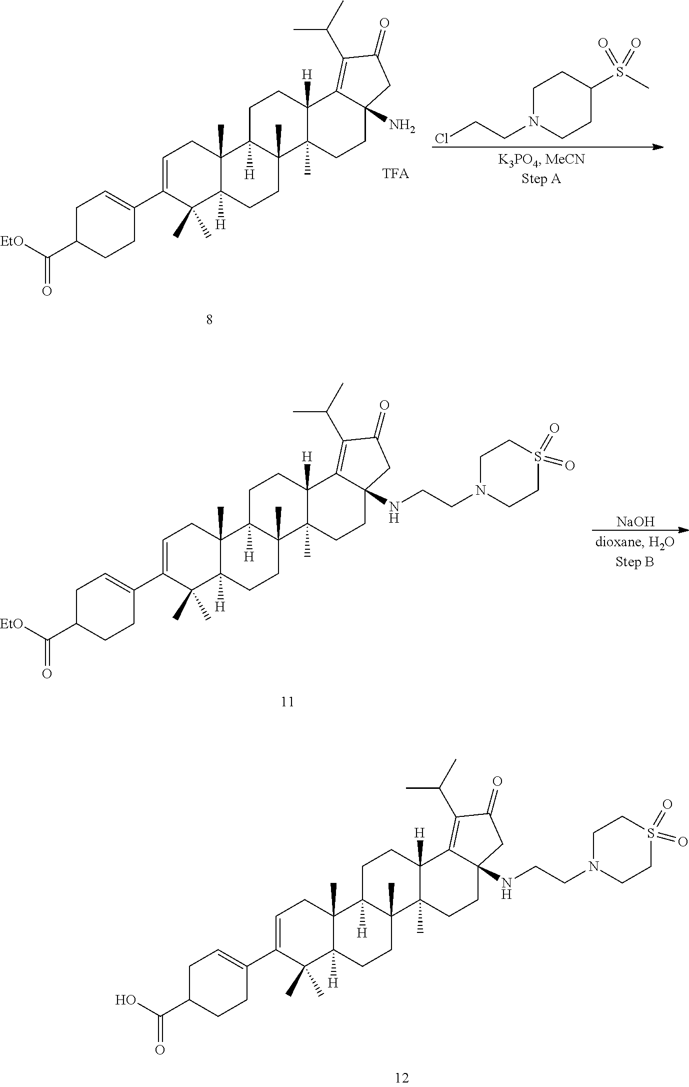

### Step A: Intermediate 11: Ethyl 4-((3aR,5aR,5bR,7aR,11aS,11bR,13aS)-3a-((2-(1,1-dioxidothiomorpholino)ethyl)amino)-1-isopropyl-5a,5b,8,8,11a-pentamethyl-2-oxo-3,3a,4,5,5a,5b,6,7,7a,8,11,11a,11b,12,13,13a-hexadecahydro-2H-cyclopenta[a]chrysen-9-yl)cyclohex-3-enecarboxylate

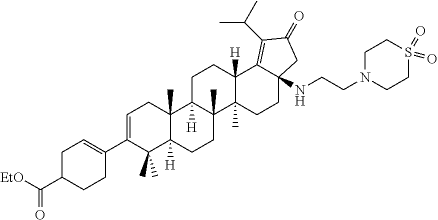

A mixture of intermediate 8 (35 mg, 0.06 mmol), 4-(2-chloroethyl)thiomorpholine 1,1-dioxide (18 mg, 0.1 mmol) and K₃PO₄ (64 mg, 0.3 mmol) in anhydrous MeCN (2.0 mL) was heated up to 100° C. and stirred overnight under N₂ atmosphere. The resulting mixture was partitioned between EtOAc and H₂O. The organic layer was washed with brine, dried over Na₂SO₄, filtered and concentrated under reduced pressure to give a residue which was purified by silica gel chromatography (0-5% MeOH/DCM) to afford intermediate 11 (8 mg, 18% yield) as a yellow oil. LC/MS: m/z calculated 736.5, found 737.5 (M+1)+.

### Step B: Compound 12: 4-((3aR,5aR,5bR,7aR,11aS,11bR,13aS)-3a-((2-(1,1-Dioxidothiomorpholino)ethyl)amino)-1-isopropyl-5a,5b,8,8,11a-pentamethyl-2-oxo-3,3a,4,5,5a,5b,6,7,7a,8,11,11a,11b,12,13,13a-hexadecahydro-2H-cyclopenta[a]chrysen-9-yl)cyclohex-3-enecarboxylic acid

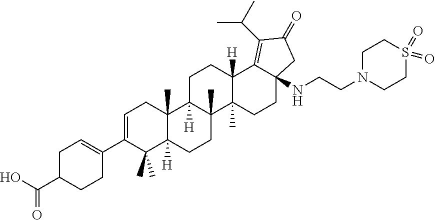

A mixture of intermediate 11 (8 mg, 0.01 mmol) in dioxane (1 mL) and 1N NaOH (0.05 mL) was stirred at 75° C. overnight. The reaction was cooled to room temperature and diluted with EtOAc then acidified with 1N HCl to pH=3-4. The layers were separated and the organic layer was washed with brine, dried over Na₂SO₄, filtered and concentrated under reduced pressure to give a residue which was purified by reverse phase chromatography (5-100% MeCN/H₂O+0.1% FA) to afford compound 12 (5 mg, 65% yield) as a white powder. LC/MS: m/z calculated 708.5, found 709.3 (M+1)+. ¹H NMR (400 MHz, DMSO) b 8.33 (s, 1H), 5.55-5.13 (m, J=91.5, 40.0 Hz, 2H), 3.18-3.00 (m, J=16.2 Hz, 6H), 2.99-2.83 (m, 4H), 2.50-0.71 (m, 51H).

### Synthesis of Intermediate 15

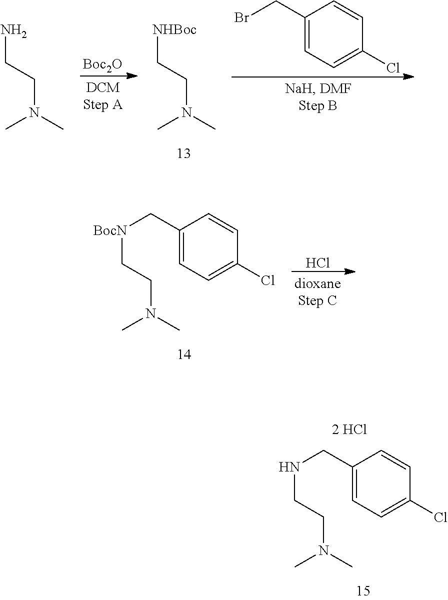

### Step A: Intermediate 13: tert-Butyl (2-(dimethylamino)ethyl)carbamate

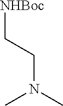

A solution of N¹,N¹-dimethylethane-1,2-diamine (2 g, 23 mmol) in DCM (30 mL) was treated with Boc₂O (5.9 g, 27 mmol). After stirring at room temperature for 1 hr, the resulting mixture was concentrated under reduced pressure to give a residue that was purified by silica age chromatography (0-10% MeOH/DCM) to afford intermediate 13 (4.2 g, 98% yield) as a colorless oil. LC/MS: m/z calculated 188.2, found 189.2 (M+1)+.

### Step B: Intermediate 14: tert-Butyl 4-chlorobenzyl(2-(dimethylamino)ethyl)carbamate

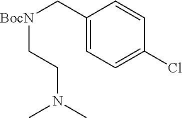 [SMILES: CN(C)CCN(Cc1ccc(Cl)cc1)C(=O)OC(C)(C)C]

At 0° C., to a solution of intermediate 13 (1 g, 5.3 mmol) in DMF (20 mL) was added NaH (60%, 255 mg, 6.4 mmol). The resulting mixture was stirred at room temperature for 1 hr, then treated with 1-(bromomethyl)-4-chlorobenzene (1.4 g, 6.9 mmol). After stirring for 30 min at room temperature, the reaction mixture was quenched with sat. NH₄Cl and extracted with EtOAc. The layers were separated and the organic layer was washed with brine, dried over Na₂SO₄, filtered and concentrated under reduced pressure to give intermediate 14 which was used in the next step without further purification. LC/MS: m/z calculated 312.2, found 313.2 (M+1)+.

### Step C: Intermediate 15: N¹-(4-Chlorobenzyl)-N²,N²-dimethylethane-1,2-diamine dihydrochloride

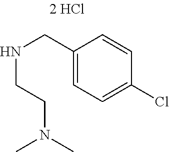

Intermediate 14 was treated with 4N HCl in dioxane (10 mL). After stirring at room temperature overnight, the reaction mixture was concentrated under reduced pressure to give a residue that was triturated with ether and filtered to afford intermediate 15 (600 mg, 40% yield over two steps, 2HCl salt) as a white solid. ¹H NMR (400 MHz, DMSO) δ 11.10 (s, 1H), 10.05 (s, 2H), 7.65 (d, J=8.4 Hz, 2H), 7.51 (d, J=8.4 Hz, 2H), 4.20 (s, 2H), 3.56-3.39 (m, 4H), 2.83 (s, 6H). LC/MS: m/z calculated 212.1, found 213.2 (M+1)+.

### Example 3 (Compound 18): 4-((3aR,5aR,5bR,7aR,11aS,11bR,13aS)-3a-((2-((4-Chlorobenzyl)(2-(dimethylamino)ethyl)amino)ethyl)amino)-1-isopropyl-5a,5b,8,8,11a-pentamethyl-2-oxo-3,3a,4,5,5a,5b,6,7,7a,8,11,11a,11b,12,13,13a-hexadecahydro-2H-cyclopenta[a]chrysen-9-yl)cyclohex-3-enecarboxylic acid

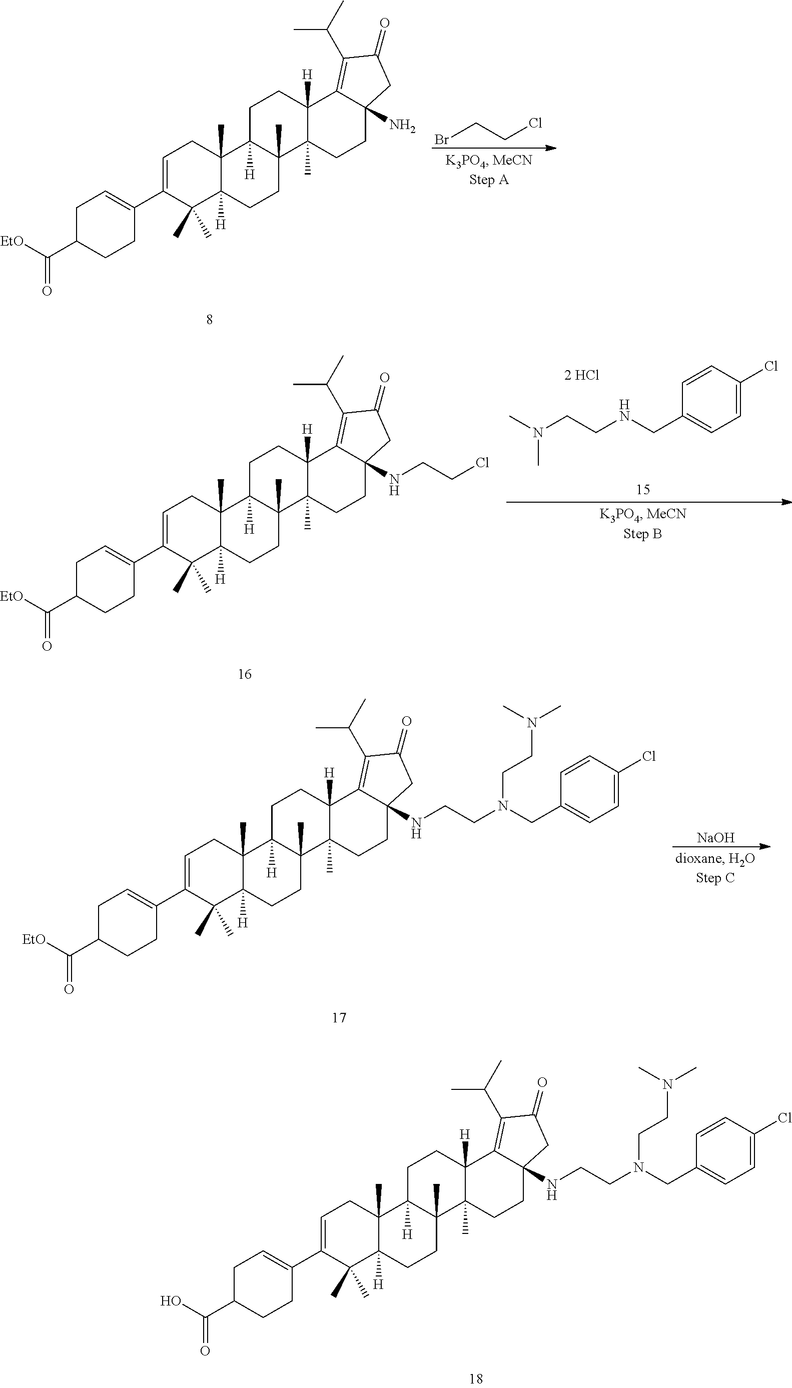

### Step A: Intermediate 16: Ethyl 4-((3aR,5aR,5bR,7aR,11aS,11bR,13aS)-3a-((2-chloroethyl)amino)-1-isopropyl-5a,5b,8,8,11a-pentamethyl-2-oxo-3,3a,4,5,5a,5b,6,7,7a,8,11,11a,11b,12,13,13a-hexadecahydro-2H-cyvclopenta[a]chrysen-9-yl)cyclohex-3-enecarboxylate

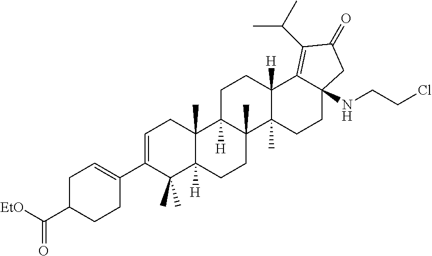

A suspension of intermediate 8 (860 mg, 1.5 mmol) and K₃PO₄ (1.9 g, 9.0 mmol) in 1-bromo-2-chloroethane (8 mL) and anhydrous MeCN (1 mL) was stirred at 100° C. overnight under N₂ atmosphere. The reaction was cooled to room temperature was partitioned between DCM and H₂O. The organic layer was washed with brine, dried over Na₂SO₄, filtered and concentrated under reduced pressure to give a residue that was purified by silica gel chromatography (0-50% EtOAc/PE) to afford intermediate 16 (200 mg, 21% yield) as a white solid. LC/MS: m/z calculated 637.4, found 638.8 (M+1)+.

### Step B: Intermediate 17: Ethyl 4-((3aR,5aR,5bR,7aR,11aS,11bR,13aS)-3a-((2-((4-chlorobenzyl)(2-(dimethylamino)ethyl)amino)ethyl)amino)-1-isopropyl-5a,5b,8,8,11a-pentamethyl-2-oxo-3,3a,4,5,5a,5b,6,7,7a,8,11,11a,11b,12,13,13a-hexadecahydro-2H-cyclopenta[a]chrysen-9-yl)cyclohex-3-enecarboxylate

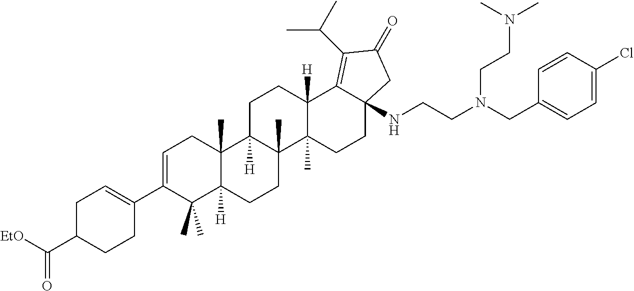

A suspension of intermediate 16 (200 mg, 0.31 mmol), intermediate 15 (167 mg, 0.78 mmol) and K₃PO₄ (532 mg, 2.5 mmol) in anhydrous MeCN (4 mL) was stirred at 100° C. overnight under N₂ atmosphere. The reaction was cooled to room temperature and was partitioned between EtOAc and H₂O. The organic layer was washed with brine, dried over Na₂SO₄, filtered and concentrated under reduced pressure to give a residue that was purified by silica gel chromatography (0-10% MeOH/DCM) to afford intermediate 17 (83 mg, 32% yield) as a white solid. LC/MS: m/z calculated 813.6, found 814.9 (M+1)+.

### Step C: Compound 18: 4-((3aR,5aR,5bR,7aR,11aS,11bR,13aS)-3a-((2-((4-Chlorobenzyl)(2-(dimethylamino)ethyl)amino)ethyl)amino)-1-isopropyl-5a,5b,8,8,11a-pentamethyl-2-oxo-3,3a,4,5,5a,5b,6,7,7a,8,11,11a,11b,12,13,13a-hexadecahydro-2H-cyclopenta[a]chrysen-9-yl)cyclohex-3-enecarboxylic acid

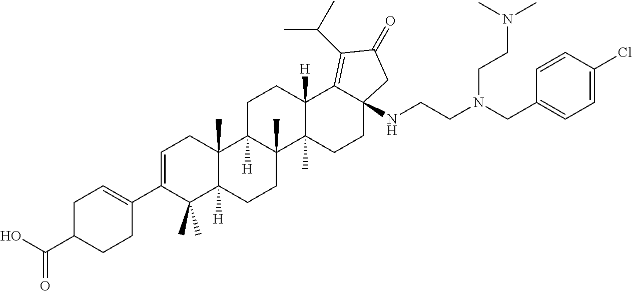

A mixture of intermediate 17 (80 mg, 0.1 mmol) in dioxane (1 mL) and 1N NaOH (1 mL) was stirred at 75° C. overnight. The reaction was cooled to room temperature and diluted with EtOAc then acidified with 1N HCl to pH=3-4. The layers were separated and the organic layer was washed with brine, dried over Na₂SO₄, filtered and concentrated under reduced pressure to give a residue which was purified by reverse phase chromatography (60-100% MeCN/H₂O+0.1% FA) to afford compound 18 (18 mg, 23% yield) as a white powder. LC/MS: m/z calculated 785.5, found 786.9 (M+1)+. ¹H NMR (400 MHz, CDCl₃ with drops of MeOD) δ 7.30-7.19 (m, 4H), 5.37 (s, 1H), 5.21 (d, J=5.5 Hz, 1H), 3.67-3.52 (m, J=7.3 Hz, 2H), 3.42-3.39 (m, 1H), 3.20-3.10 (m, 1H), 2.95 (d, J=12.2 Hz, 1H), 2.61-0.91 (m, 59H).

### Synthesis of Intermediate 20

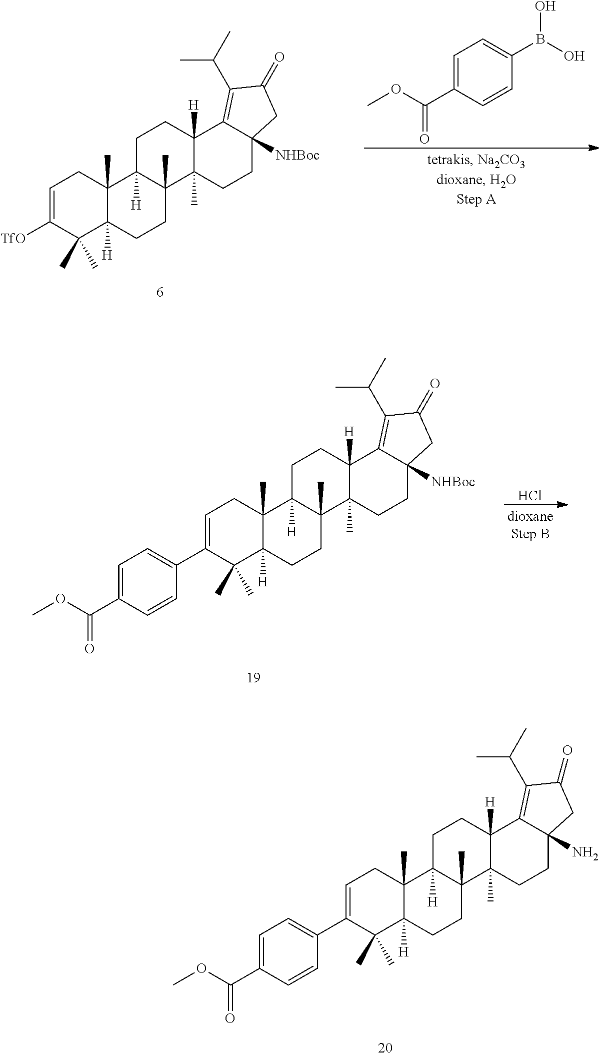

### Step A: Intermediate 19: Methyl 4-((3aR,5aR,5bR,7aR,11aS,11bR,13aS)-3a-((tert-butoxycarbonyl)amino)-1-isopropyl-5a,5b,8,8,11a-pentamethyl-2-oxo-3,3a,4,5,5a,5b,6,7,7a,8,11,11a,11b,12,13,13a-hexadecahydro-2H-cyclopenta[a]chrysen-9-yl)benzoate

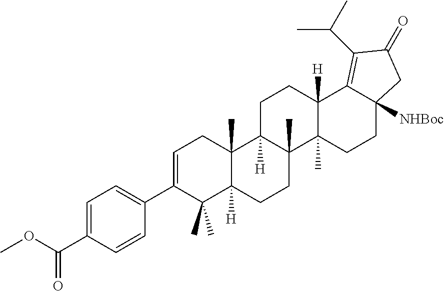

A mixture of intermediate 6 (1.5 g, 2.2 mmol), (4-(methoxycarbonyl)phenyl)boronic acid (603 mg, 3.3 mmol), tetrakis (516 mg, 0.44 mmol) and Na₂CO₃ (710 mg, 6.7 mmol) in dioxane (10 mL) and H₂O (2.5 mL) was purged with N₂ three times. The reaction was stirred at 85° C. overnight, cooled to room temperature, filtered through a pad of Celite, and the filtrate was partitioned between EtOAc and H₂O. The organic layer was washed with brine, dried over Na₂SO₄, filtered and concentrated under reduced pressure to give a residue that was purified by silica gel chromatography (0-40% EtOAc/PE) to afford intermediate 19 (700 mg, 48% yield) as a white solid. LC/MS: m/z calculated 657.4, found 658.8 (M+1)+.

### Step B: Intermediate 20: Methyl 4-((3aR,5aR,5bR,7aR,11aS,11bR,13aS)-3a-amino-1-isopropyl-5a,5b,8,8,11a-pentamethyl-2-oxo-3,3a,4,5,5a,5b,6,7,7a,8,11,11a,11b,12,13,13a-hexadecahydro-2H-cyclopenta[a]chrysen-9-yl)benzoate hydrochloride

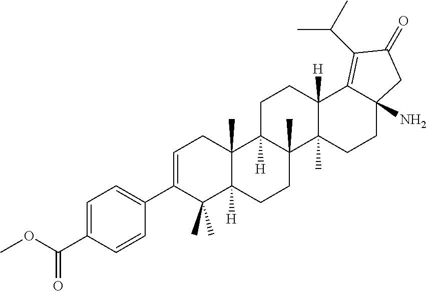

A mixture of intermediate 19 (700 mg, 1.1 mmol) in 4N HCl/dioxane (7 mL) and DCM (7 mL) was stirred at room temperature overnight. The resulting mixture was concentrated under reduced pressure to afford intermediate 20 (873 mg, quant. yield) as a white solid which was used in the next step without further purification. LC/MS: m/z calculated 557.4, found 558.7 (M+1)+.

### Example 4 (Compound 21): 4-((3aR,5aR,5bR,7aR,11aS,11bR,13aS)-3a-((2-((4-Chlorobenzyl)(2-(dimethylamino)ethyl)amino)ethyl)amino)-1-isopropyl-5a,5b,8,8,11a-pentamethyl-2-oxo-3,3a,4,5,5a,5b,6,7,7a,8,11,11a,11b,12,13,13a-hexadecahydro-2H-cyclopenta[a]chrysen-9-yl)benzoic acid hydrochloride salt

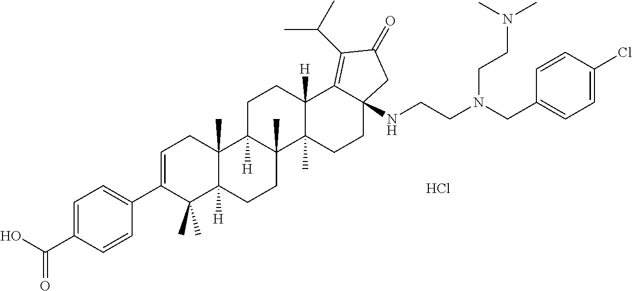

The title compound, compound 21, was made in a manner similar to Example 3 using intermediate 20. The crude product was purified by reverse phase chromatography (50-100% MeCN/H₂O+0.1% FA) and then treated with a few drops of HCl in dioxanes to afford compound 21 (32 mg, 36% yield) as a white powder. LC/MS: m/z calculated 781.5, found 782.7 (M+1)+. ¹H NMR (400 MHz, CDCl₃ with drops of MeOD) δ 7.94 (d, J=7.9 Hz, 2H), 7.32-7.13 (m, J=24.7, 16.6, 8.3 Hz, 6H), 5.32 (d, J=5.0 Hz, 1H), 3.61 (q, J=13.7 Hz, 2H), 3.27-3.07 (m, 10H), 3.01-2.57 (m, 7H), 2.47-0.94 (m, 38H).

### Example 5 (Compound 22): 4-((3aR,5aR,5bR,11aS)-3a-((2-(1,1-Dioxidothiomorpholino)ethyl)amino)-1-isopropyl-5a,5b,8,8,11a-pentamethyl-2-oxo-3,3a,4,5,5a,5b,6,7,7a,8,11,11a,11b,12,13,13a-hexadecahydro-2H-cyclopenta[a]chrysen-9-yl)benzoic acid hydrochloride

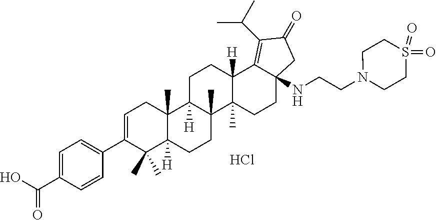

The title compound, compound 22, was made in a manner similar to Example 2 using intermediate 20. The crude product was purified by reverse phase chromatography (50-100% MeCN/H₂O+0.1% FA) and then treated with a few drops of HCl in dioxanes to afford compound 22 (31 mg, 45% yield) as a white powder. LC/MS: m/z calculated 704.4, found 705.2 (M+1)+. ¹H NMR (400 MHz, CDCl₃ with drops of MeOD) b 7.96 (d, J=8.2 Hz, 2H), 7.21 (d, J=8.2 Hz, 2H), 5.32 (d, J=4.9 Hz, 1H), 3.47-3.15 (m, 10H), 2.69-0.90 (m, 43H).

### Synthesis of Intermediate 24

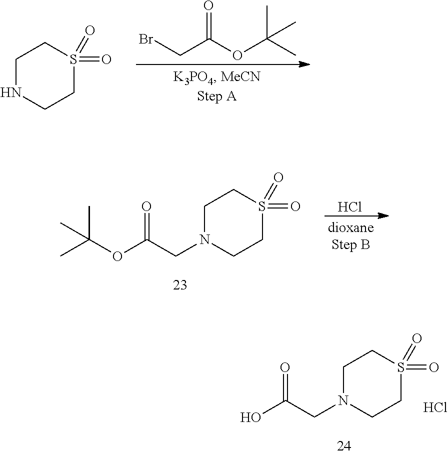

### Step A: Intermediate 23: tert-Butyl 2-(1,1-dioxidothiomorpholino)acetate

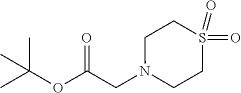 [SMILES: CC(C)(C)OC(=O)CN1CCS(=O)(=O)CC1]

A mixture of thiomorpholine 1,1-dioxide (500 mg, 3.7 mmol), tert-butyl 2-bromoacetate (1.1 g, 5.5 mmol), K₃PO₄ (2.5 g, 1.07 mmol) in anhydrous MeCN (5 mL) was stirred at 85° C. overnight under N₂ atmosphere. The resulting mixture was partitioned between EtOAc and H₂O. The organic layer was washed with brine, dried over Na₂SO₄, filtered and concentrated under reduced pressure to give a residue that was purified by silica gel chromatography (0-50% EtOAc/PE) to afford intermediate 23 (485 mg, 53% yield) as a colorless oil. LC/MS: m/z calculated 249.1, found 250.4 (M+1)+.

### Step B: Intermediate 24: 2-(1,1-Dioxidothiomorpholino)acetic acid hydrochloride salt

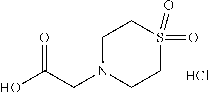 [SMILES: O=C(O)CN1CCS(=O)(=O)CC1 ; Cl]

A mixture of intermediate 23 (485 mg, 1.9 mmol) in 4N HCl/dioxane (5 mL) and DCM (5 mL) was stirred at room temperature overnight. The resulting mixture was concentrated under reduced pressure to give intermediate 24 (432 mg, quant. yield) as a white solid which was used without further purification. LC/MS: m/z calculated 193.0, found 194.0 (M+1)+.

### Example 6 (Compound 26): 4-((3aR,5aR,5bR,11aS)-3a-(2-(1,1-Dioxidothiomorpholino)acetamido)-1-isopropyl-5a,5b,8,8,11a-pentamethyl-2-oxo-3,3a,4,5,5a,5b,6,7,7a,8,11,11a,11b,12,13,13a-hexadecahydro-2H-cyclopenta[a]chrysen-9-yl)benzoic acid hydrochloride salt

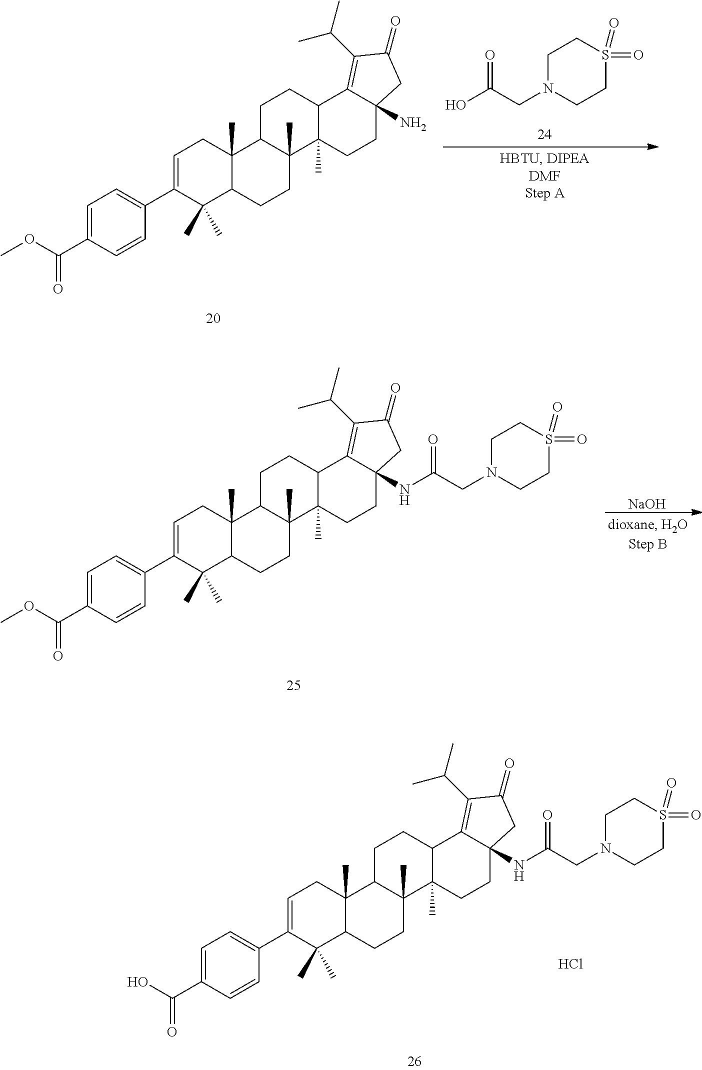

### Step A: Intermediate 25: Methyl 4-((3aR,5aR,5bR,11aS)-3a-(2-(1,1-dioxidothiomorpholino)acetamido)-1-isopropyl-5a,5b,8,8,11a-pentamethyl-2-oxo-3,3a,4,5,5a,5b,6,7,7a,8,11,11a,11b,12,13,13a-hexadecahydro-2H-cyclopenta[a]chrysen-9-yl)benzoate

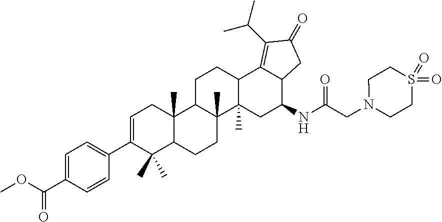

A mixture of intermediate 20 (100 mg, 0.18 mmol), intermediate 24, HBTU (104 mg, 0.27 mg) and DIPEA (0.23 mL, 1.25 mmol) in DCM (2 mL) was stirred at room temperature overnight. The resulting mixture was partitioned between DCM and H₂O. The organic layer was washed with brine, dried over Na₂SO₄, filtered and concentrated under reduced pressure to give a reside that was purified by silica gel chromatography (0-10% MeOH/DCM) to afford intermediate 25 (70 mg, 53% yield) as a white solid. LC/MS: m/z calculated 732.4, found 733.5 (M+1)+.

### Step B: Compound 26: 4-((3aR,5aR,5bR,11aS)-3a-(2-(1,1-Dioxidothiomorpholino)acetamido)-1-isopropyl-5a,5b,8,8,11a-pentamethyl-2-oxo-3,3a,4,5,5a,5b,6,7,7a,8,11,11a,11b,12,13,13a-hexadecahydro-2H-cyclopenta[a]chrysen-9-yl)benzoic acid hydrochloride salt

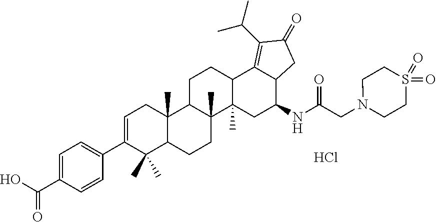

The title compound, compound 26, was made in a manner similar to Example 1, step B, using intermediate 25 (100 mg, 0.14 mmol). The crude residue was purified by reverse phase chromatography (50-100% MeCN/H₂O+0.1% FA) and the treated with a few drops of HCl in dioxanes to afford compound 26 (37 mg, 38% yield) as a white powder. LC/MS: m/z calculated 718.4, found 719.2 (M+1)+. ¹H NMR (400 MHz, CDCl₃ with drops of MeOD) b 7.96 (d, J=8.2 Hz, 2H), 7.21 (d, J=8.2 Hz, 2H), 5.33 (d, J=5.0 Hz, 1H), 3.74-3.40 (m, 8H), 3.19 (dt, J=13.9, 6.9 Hz, 1H), 2.90-2.79 (m, 1H), 2.66 (d, J=18.4 Hz, 1H), 2.34-0.89 (m, 40H).

### Example 7 (Compound 29)

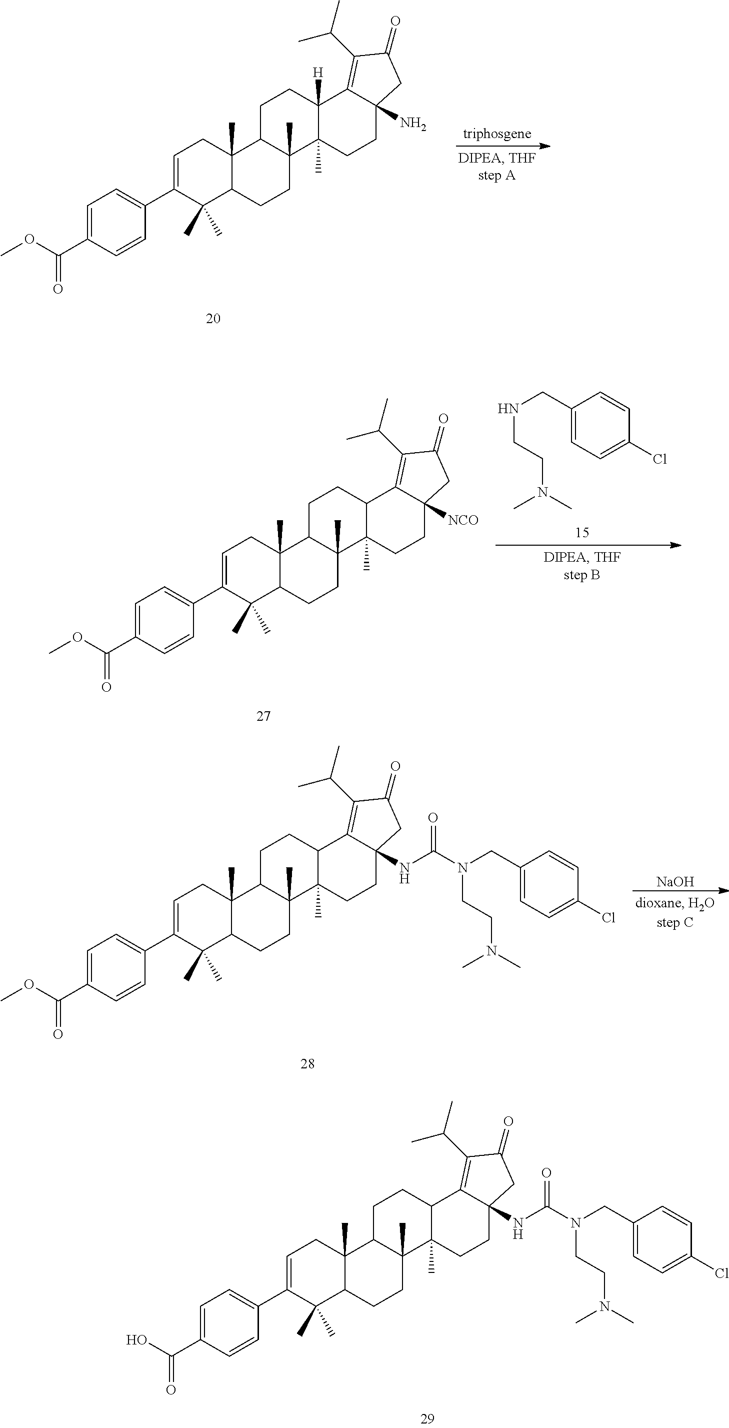

### Step A: Intermediate 27: Methyl 4-((3aR,5aR,5bR,11aS)-3a-isocyanato-1-isopropyl-5a,5b,8,8,11a-pentamethyl-2-oxo-3,3a,4,5,5a,5b,6,7,7a,8,11,11a,11b,12,13,13a-hexadecahydro-2H-cyvclopenta[a]chrysen-9-yl)benzoate

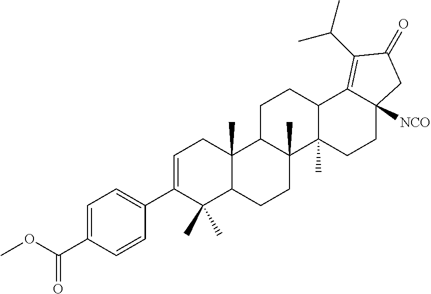

At 0° C., to a solution of methyl 4-((3aR,5aR,5bR,11aS)-3a-amino-1-isopropyl-5a,5b,8,8,11a-pentamethyl-2-oxo-3,3a,4,5,5a,5b,6,7,7a,8,11,11a,11b,12,13,13a-hexadecahydro-2H-cyclopenta[a]chrysen-9-yl)benzoate (120 mg, 0.215 mmol) and DIPEA (0.095 mL, 0.538 mmol) in THF (1.2 mL) was added triphosgene (127 mg, 0.43 mmol). After stirred at r.t. for 1 hr, the resulting mixture was quenched with H₂O and extracted with EtOAc. The organic layer was washed with brine, dried over Na₂SO₄, filtered and concentrated under reduced pressure to give the crude product which was purified by flash chromatography (silica gel, 0-10% EtOAc in PE) to afford the title compound (91 mg, 72% yield) as a white solid. LC-MS (ESI): m/z (M+1)=584.3.

### Step B: Intermediate 28: Methyl 4-((3aR,5aR,5bR,11aS)-3a-(3-(4-chlorobenzyl)-3-(2-(dimethylamino)ethyl)ureido)-1-isopropyl-5a,5b,8,8,11a-pentamethyl-2-oxo-3,3a,4,5,5a,5b,6,7,7a,8,11,11a,11b,12,13,13a-hexadecahydro-2H-cyclopenta[a]chrysen-9-yl)benzoate

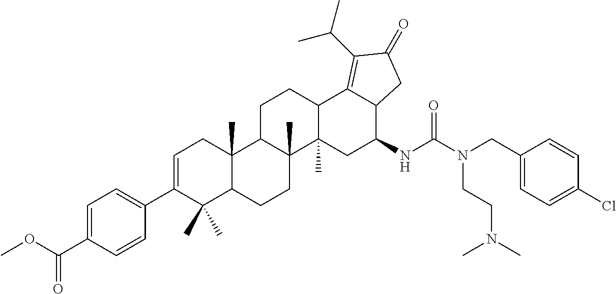

A solution of methyl 4-((3aR,5aR,5bR,11aS)-3a-isocyanato-1-isopropyl-5a,5b,8,8,11a-pentamethyl-2-oxo-3,3a,4,5,5a,5b,6,7,7a,8,11,11a,11b,12,13,13a-hexadecahydro-2H-cyclopenta[a]chrysen-9-yl)benzoate (91 mg, 0.156 mmol), N¹-(4-chlorobenzyl)-N²,N²-dimethylethane-1,2-diamine (133 mg, 0.468 mmol) and DIPEA (0.21 mL, 1.25 mmol) in THF (1 mL) was stirred at r.t. overnight. The resulting mixture was partitioned between EtOAc and H₂O. The organic layer was washed with brine, dried over Na₂SO₄, filtered and concentrated under reduced pressure to give the crude product which was purified by flash chromatography (silica gel, 0-10% MeOH in DCM) to afford the title compound (70 mg, 57% yield) as a white solid. LC-MS (ESI): m/z (M/M+2)=796.5/798.5.

### Step C: Compound 29: 4-((3aR,5aR,5bR,11aS)-3a-(3-(4-chlorobenzyl)-3-(2-(dimethylamino)ethyl) ureido)-1-isopropyl-5a,5b,8,8,11a-pentamethyl-2-oxo-3,3a,4,5,5a,5b,6,7,7a,8,11,11a,11b,12,13,13a-hexadecahydro-2H-cyclopenta[a]chrysen-9-yl)benzoic acid

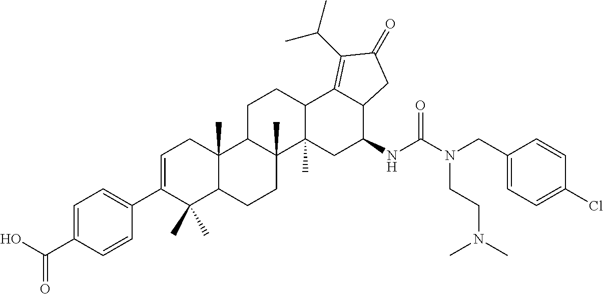

A mixture of methyl 4-((3aR,5aR,5bR,11 aS)-3a-(3-(4-chlorobenzyl)-3-(2-(dimethyl amino)ethyl)ureido)-1-isopropyl-5a,5b,8,8,11a-pentamethyl-2-oxo-3,3a,4,5,5a,5b,6,7,7a,8,11,11a,11b,12,13,13a-hexadecahydro-2H-cyclopenta[a]chrysen-9-yl)benzoate (70 mg, 0.088 mmol) in 1N NaOH (0.88 mL) and dioxane (1 mL) was stirred at 75° C. for 1.5 hr. The resulting mixture was acidified with 1N HCl to pH 3-4 and extracted with EtOAc. The organic layer was washed with brine, dried over Na₂SO₄, filtered and concentrated under reduced pressure to give the crude product which was purified by HPLC (C18, 50-100% MeCN in H₂O with 0.1% formic acid) to afford the title compound (38 mg, 53% yield) as a white powder after lyophilization (A few drops of HCl in dioxane was added to the fraction collected by LCMS to give the HCl salt). LC-MS (ESI): m/z (M/M+2)=782.4/784.4. ¹H NMR (400 MHz, CDCl₃ with drops of MeOD) δ 7.95 (d, J=8.2 Hz, 2H), 7.37 (d, J=8.3 Hz, 2H), 7.20 (t, J=8.9 Hz, 4H), 5.32 (d, J=4.4 Hz, 1H), 4.60 (dd, J=59.7, 17.4 Hz, 2H), 4.05-3.91 (m, 1H), 3.68 (dd, J=14.1, 7.0 Hz, 1H), 3.60-3.48 (m, 1H), 3.42-3.34 (m, 2H), 3.29-3.11 (m, 3H), 2.95-2.59 (m, 8H), 2.40-0.52 (m, 35H).

### Example 8 (Compound 33)

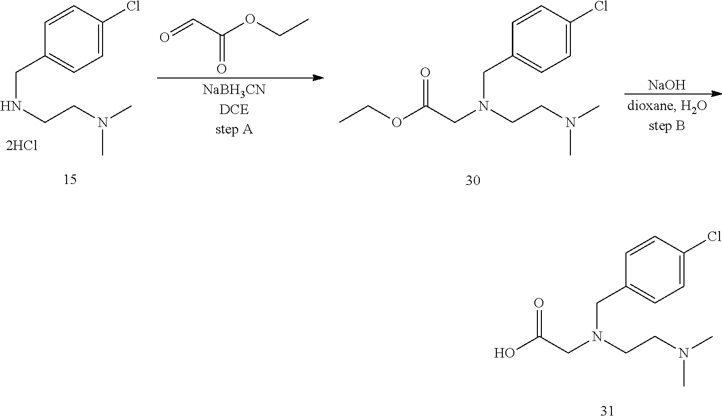

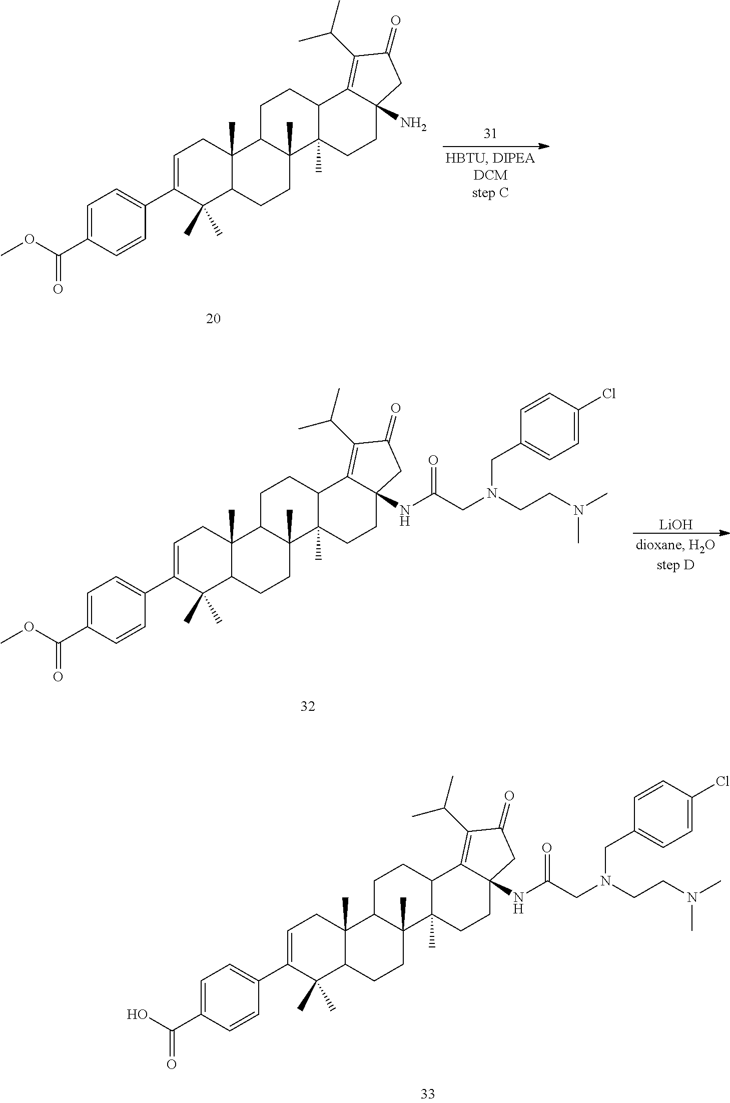

### Step A: Intermediate 30: Ethyl N-(4-chlorobenzyl)-N-(2-(dimethylamino)ethyl)glycinate

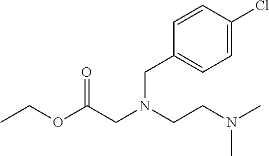 [SMILES: CCOC(=O)CN(CCN(C)C)Cc1ccc(Cl)cc1]

A mixture of N¹-(4-chlorobenzyl)-N²,N²-dimethylethane-1,2-diamine (800 mg, 3.76 mmol), ethyl 2-oxoacetate (50% toluene solution, 422 mg, 4.14 mmol) and NaBH₃CN (331 mg, 5.27 mmol) in DCE (8 mL) was stirred at r.t. for 4 hr. The resulting mixture was quenched with sat. NH₄Cl aq. solution and extracted with DCM/i-PrOH (85:15). The organic layer was washed with brine, dried over Na₂SO₄, filtered and concentrated under reduced pressure to give the title compound (200 mg, 18% yield) as a yellow oil which was used in the next step without further purification. LC-MS (ESI): m/z (M/M+2)=299.3/301.4.

### Step B: Intermediate 31: N-(4-chlorobenzyl)-N-(2-(dimethylamino)ethyl)glycine

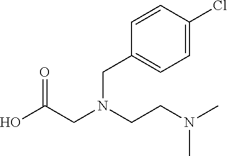 [SMILES: CN(C)CCN(CC(=O)O)Cc1ccc(Cl)cc1]

A mixture of ethyl N-(4-chlorobenzyl)-N-(2-(dimethylamino)ethyl)glycinate (200 mg, 0.67 mmol) in 10N NaOH (0.67 mL, 6.7 mmol) and dioxane (1 mL) was stirred at 120° C. overnight. The resulting mixture was neutralized with 1N HCl and concentrated under reduced pressure to give the crude product which was purified by HPLC (C18, 0-100% MeOH in H₂O with 0.3% formic acid) to afford the title compound (65 mg, 36% yield) as a colorless oil. LC-MS (ESI): m/z (M/M+2)=271.2/273.1.

### Step C: Intermediate 32: Methyl 4-((3aR,5aR,5bR,11 aS)-3a-(2-((4-chlorobenzyl) (2-(dimethylamino)ethyl)amino)acetamido)-1-isopropyl-5a,5b,8,8,11a-pentamethyl-2-oxo-3,3a, 4,5,5a,5b,6,7,7a,8,11,11a,11b,12,13,13a-hexadecahydro-2H-cyclopenta[a]chrysen-9-yl)benzoate

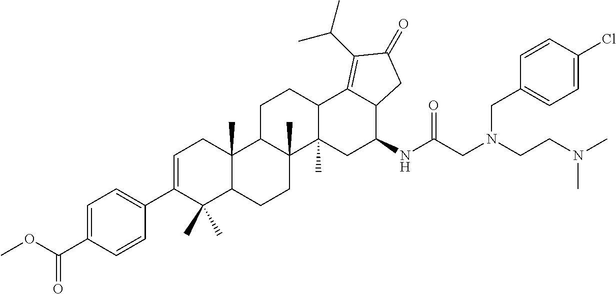

A mixture of methyl 4-((3aR,5aR,5bR,11aS)-3a-amino-1-isopropyl-5a,5b,8,8,11a-pentamethyl-2-oxo-3,3a,4,5,5a,5b,6,7,7a,8,11,11a,11b,12,13,13a-hexadecahydro-2H-cyclopenta[a]chrysen-9-yl)benzoate (60 mg, 0.108 mmol), N-(4-chlorobenzyl)-N-(2-(dimethylamino)ethyl)glycine (116 mg, 0.43 mmol), HBTU (61 mg, 0.16 mmol) and DIPEA (76.3 mg, 0.592 mmol) in DCM (1 mL) was stirred at r.t. overnight. The resulting mixture was partitioned between DCM and H₂O. The organic layer was washed with brine, dried over Na₂SO₄, filtered and concentrated under reduced pressure to give the crude product which was purified by flash chromatography (silica gel, 0-10% MeOH in DCM) to afford the title compound (60 mg, 69% yield) as a white solid. LC-MS (ESI): m/z (M/M+2)=810.5/812.5.

### Step C: Compound 33: 4-((3aR,5aR,5bR,11aS)-3a-(2-((4-chlorobenzyl)(2-(dimethylamino)ethyl)amino)acetamido)-1-isopropyl-5a,5b,8,8,11a-pentamethyl-2-oxo-3,3a, 4,5,5a,5b,6,7,7a,8,11,11a,11b,12,13,13a-hexadecahydro-2H-cyclopenta[a]chrysen-9-yl)benzoic acid

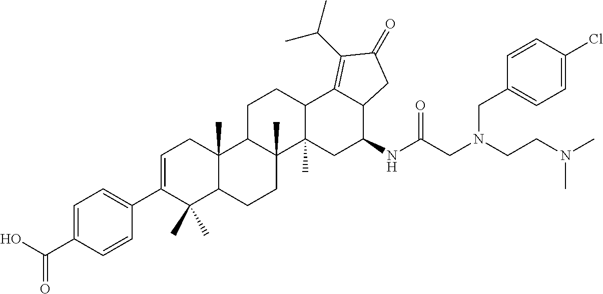

A mixture of methyl 4-((3aR,5aR,5bR,11 aS)-3a-(2-((4-chlorobenzyl)(2-(dimethylamino)ethyl)amino)acetamido)-1-isopropyl-5a,5b,8,8,8,11a-pentamethyl-2-oxo-3,3a,4,5,5a,5b,6,7,7a,8,11,11a,11 b,12,13,13a-hexadecahydro-2H-cyclopenta[a]chrysen-9-yl)benzoate (60 mg, 0.074 mmol) in 2.5N LiOH (1 mL) and dioxane (1 mL) was stirred at 75° C. for 2.5 hr. The resulting mixture was acidified with 1N HCl to pH 3-4 and extracted with EtOAc. The organic layer was washed with brine, dried over Na₂SO₄, filtered and concentrated under reduced pressure to give the crude product which was purified by HPLC (C18, 50-100% MeCN in H₂O with 0.1% formic acid) to afford the title compound (8.2 mg, 14% yield) as a white powder after lyophilization (A few drops of HCl in dioxane was added to the fraction collected by LCMS to give the HCl salt). LC-MS (ESI): m/z (M/M+2)=796.5/798.4. ¹H NMR (400 MHz, CDCl₃ with drops of MeOD) (7.95 (d, J=8.1 Hz, 2H), 7.60 (d, J=7.4 Hz, 2H), 7.41 (d, J=7.9 Hz, 2H), 7.21 (d, J=8.1 Hz, 2H), 5.32 (d, J=5.0 Hz, 1H), 4.30 (s, 2H), 4.15-3.27 (m, 8H), 3.18 (dt, J=14.1, 7.2 Hz, 1H), 2.84 (s, 6H), 2.63-2.32 (m, 6H), 2.18-0.95 (m, 32H).

### Example 9 (Compound 37)

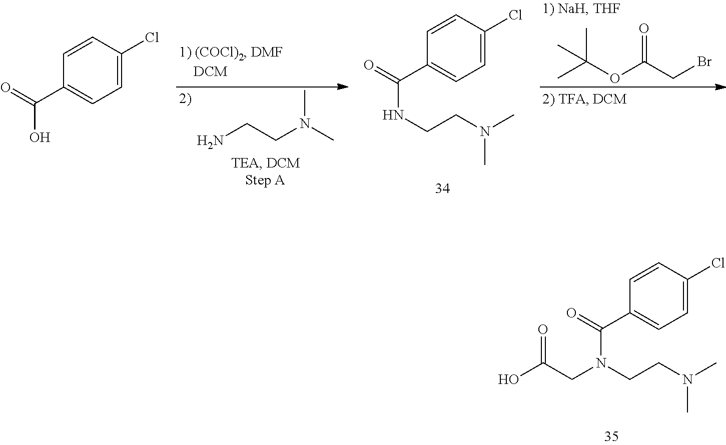

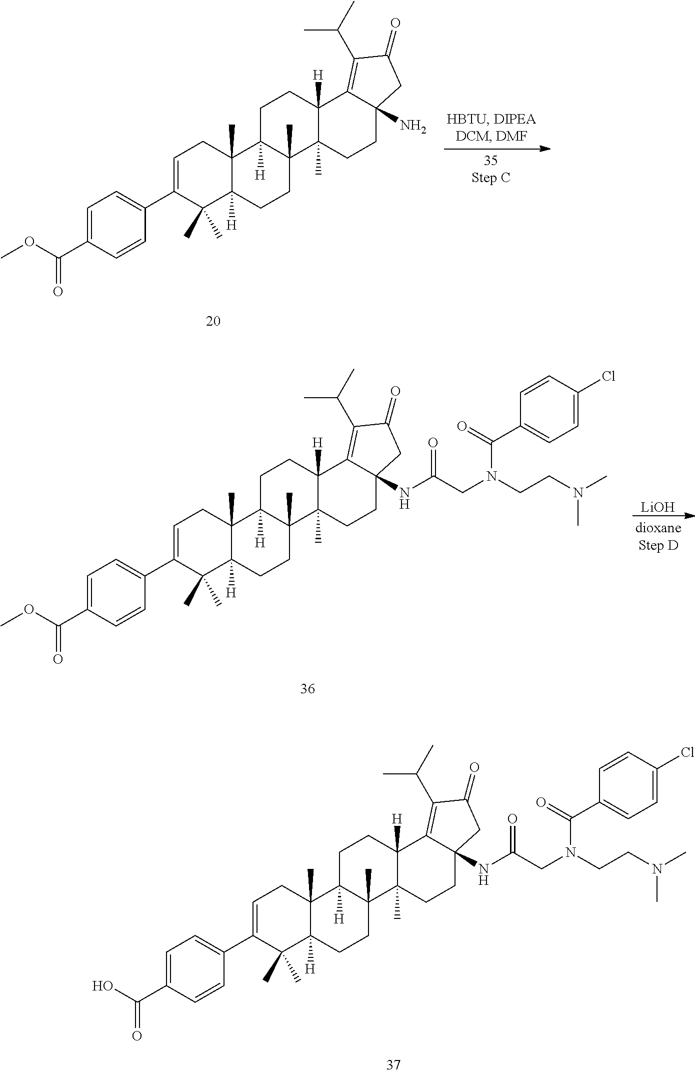

### Step A: Intermediate 34: 4-chloro-N-(2-(dimethylamino)ethyl)benzamide

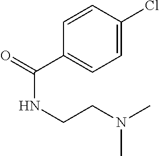

At 0° C., to a solution of 4-chlorobenzoic acid (1 g, 6.4 mmol) in DCM (20 mL) was added oxalyl chloride (10 mL) and DMF (one drop). After warmed up to r.t. and stirred for 30 min, the resulting mixture was concentrated under reduced pressure to give the crude acyl chloride which was diluted with DCM (10 mL) and introduced to a pre-cold solution of M,M-dimethylethane-1,2-diamine (0.62 g, 7.0 mmol) and TEA (0.97 g, 9.6 mmol) in DCM. After stirred at r.t. for 2 hr, the resulting mixture was quenched with sat. NaHCO₃ aq. solution and extracted with DCM. The organic layer was washed with brine, dried over Na₂SO₄, filtered and concentrated under reduced pressure to give the crude product which was purified by ISCO (silica gel, 0-10% MeOH in DCM) to afford 4-chlorobenzoyl chloride (1.3 g, 90% yield) as a yellow oil. LC-MS (ESI): m/z (M/M+2)=227.3/229.3. ¹H NMR (400 MHz, CDCl₃) b 7.73 (d, J=8.6 Hz, 2H), 7.39 (d, J=8.6 Hz, 2H), 6.83 (s, 1H), 3.50 (dd, J=11.3, 5.4 Hz, 2H), 2.59-2.47 (m, 2H), 2.27 (s, 6H).

### Step B: Intermediate 35: 2-(4-c]Chloro-N-(2-(dimethylamino)ethyl)benzamido)acetic acid

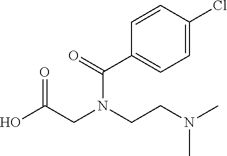 [SMILES: CN(C)CCN(CC(=O)O)C(=O)c1ccc(Cl)cc1]

At 0° C., to a solution of 4-chloro-N-(2-(dimethylamino)ethyl)benzamide (1.3 g, 5.75 mmol) in THF (20 mL) was added NaH (277 mg, 6.9 mmol). The resulting mixture was warmed up to r.t. and stirred for 30 min before the addition of tert-butyl 2-bromoacetate (1.24 g, 6.3 mmol). After stirred at r.t. overnight, the reaction mixture was quenched with sat. NH₄Cl aq. solution and extracted with EtOAc. The organic layer was washed with brine, dried over Na₂SO₄, filtered and concentrated under reduced pressure to give the crude product which was purified by reverse phase chromatography (C18, 0-100% MeCN in H₂O with 0.1% formic acid) to afford tert-butyl 2-(4-chloro-N-(2-(dimethylamino)ethyl)benzamido)acetate which was dissolved in DCM (10 mL) and treated with TFA (5 mL) to give 2-(4-chloro-N-(2-(dimethylamino)ethyl)benzamido)acetic acid (280 mg, 17% yield) as a light yellow solid. LC-MS (ESI): m/z (M/M+2)=285.0/287.0. ¹H NMR (400 MHz, DMSO) δ 7.49 (d, J=8.4 Hz, 2H), 7.39 (d, J=8.4 Hz, 2H), 3.83-3.73 (m, 2H), 3.67 (s, 2H), 3.25-3.11 (m, J=6.7, 3.7 Hz, 2H), 2.71 (s, 6H). (the acid proton was not observed)

### Step C: Intermediate 36: Methyl 4-((3aR,5aR,5bR,7aR,11aS,11bR,13aS)-3a-(2-(4-chloro-N-(2-(dimethylamino)ethyl)benzamido)acetamido)-1-isopropyl-5a,5b,8,8,11a-pentamethyl-2-oxo-3,3a,4,5,5a,5b,6,7,7a,8,11,11a,11b,12,13,13a-hexadecahydro-2H-cyclopenta[a]chrysen-9-yl)benzoate

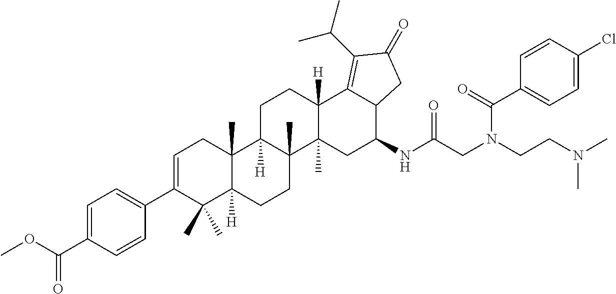

A mixture of methyl 4-((3aR,5aR,5bR,7aR,11aS,11bR,13aS)-3a-amino-1-isopropyl-5a,5b,8,8,11a-pentamethyl-2-oxo-3,3a,4,5,5a,5b,6,7,7a,8,11,11a,11b,12,13,13a-hexadecahydro-2H-cyclopenta[a]chrysen-9-yl)benzoate (80 mg, 0.14 mmol), 2-(4-c]Chloro-N-(2-(dimethylamino)ethyl)benzamido)acetic acid (100 mg, 0.35 mmol), HBTU (250 mg, 0.65 mmol) and DIPEA (270 mg, 2.1 mmol) in DCM (5 mL) and DMF (2 mL) was stirred at 40° C. overnight. The resulting mixture was quenched with sat. NaHCO₃ aq. solution and extracted with DCM. The organic layer was washed with brine, dried over Na₂SO₄, filtered and concentrated under reduced pressure to give the crude product which was purified by ISCO (silica gel, 0-10% MeOH in DCM) to afford methyl 4-((3aR,5aR,5bR,7aR,11aS,11bR,13aS)-3a-(2-(4-chloro-N-(2-(dimethylamino)ethyl)benzamido)acetamido)-1-isopropyl-5a,5b,8,8,11a-pentamethyl-2-oxo-3,3a,4,5,5a,5b,6,7,7a,8,11,11a,11b,12,13,13a-hexadecahydro-2H-cyclopenta[a]chrysen-9-yl)benzoate (40 mg, 34% yield) as light yellow solid. LC-MS (ESI): m/z (M/M+2)=824.2/826.4. ¹H NMR (400 MHz, CDCl₃) δ 7.94 (d, J=8.3 Hz, 2H), 7.69 (s, 1H), 7.51-7.33 (m, 4H), 7.21 (d, J=8.3 Hz, 2H), 5.38-5.23 (m, 3H), 4.20-3.95 (m, J=15.6 Hz, 2H), 3.91 (s, 3H), 3.71-3.57 (m, 1H), 3.56-3.36 (m, 1H), 3.23-3.03 (m, 2H), 2.63-1.00 (m, 45H).

### Step D: Compound 37: 4-((3aR,5aR,5bR,7aR,11aS,11bR,13aS)-3a-(2-(4-Chloro-N-(2-(dimethylamino)ethyl)benzamido)acetamido)-1-isopropyl-5a,5b,8,8,11a-pentamethyl-2-oxo-3,3a,4,5,5a,5b,6,7,7a,8,11,11a,11b,12,13,13a-hexadecahydro-2H-cyclopenta[a]chrysen-9-yl)benzoic acid

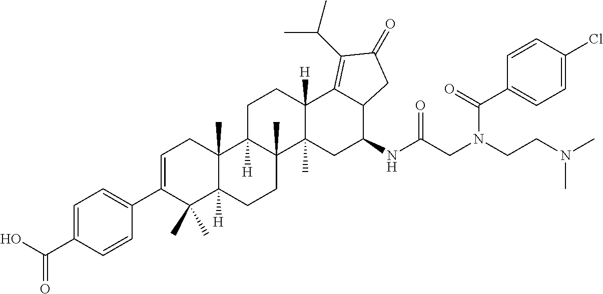

A mixture of methyl 4-((3aR,5aR,5bR,7aR,11aS,11bR,13aS)-3a-(2-(4-chloro-N-(2-(dimethylamino)ethyl)benzamido)acetamido)-1-isopropyl-5a,5b,8,8,11a-pentamethyl-2-oxo-3,3a,4,5,5a,5b,6,7,7a,8,11,11a,11b,12,13,13a-hexadecahydro-2H-cyclopenta[a]chrysen-9-yl)benzoate (40 mg, 0.05 mmol) in 2.5N LiOH (0.4 mL) and dioxane (2 mL) was stirred at 60° C. for 2 hr. The resulting mixture was acidified with 1N HCl to pH 6-7 and extracted with DCM. The organic layer was washed with brine, dried over Na₂SO₄, filtered and concentrated under reduced pressure to give the crude product which was purified by reverse phase chromatography (C18, 0-100% MeCN in H₂O with 0.1% formic acid) to afford 4-((3aR,5aR,5bR,7aR,11aS,11 bR,13aS)-3a-(2-(4-Chloro-N-(2-(dimethylamino)ethyl)benzamido)acetamido)-1-isopropyl-5a,5b,8,8,11a-pentamethyl-2-oxo-3,3a,4,5,5a,5b,6,7,7a,8,11,111a,11b,12,13,13a-hexadecahydro-2H-cyclopenta[a]chrysen-9-yl)benzoic acid (20 mg, 51% yield) as a white powder. A few drops of 4N HCl in dioxane was added to the fraction collected by HPLC to give the corresponding HCl salt. LC-MS (ESI): m/z (M/M+2)=810.5/812.4. ¹H NMR (400 MHz, MeOD) b 7.93 (d, J=8.3 Hz, 2H), 7.47 (dd, J=26.7, 8.2 Hz, 4H), 7.24 (d, J=8.2 Hz, 2H), 5.34 (d, J=4.8 Hz, 1H), 4.21-3.87 (m, 4H), 3.44-3.34 (m, 2H), 3.28-3.20 (m, 1H), 2.99 (d, J=9.1 Hz, 6H), 2.80-2.44 (m, 3H), 2.38-2.15 (m, 3H), 2.04-0.96 (m, 34H).

### Example 10 (Compound 51)

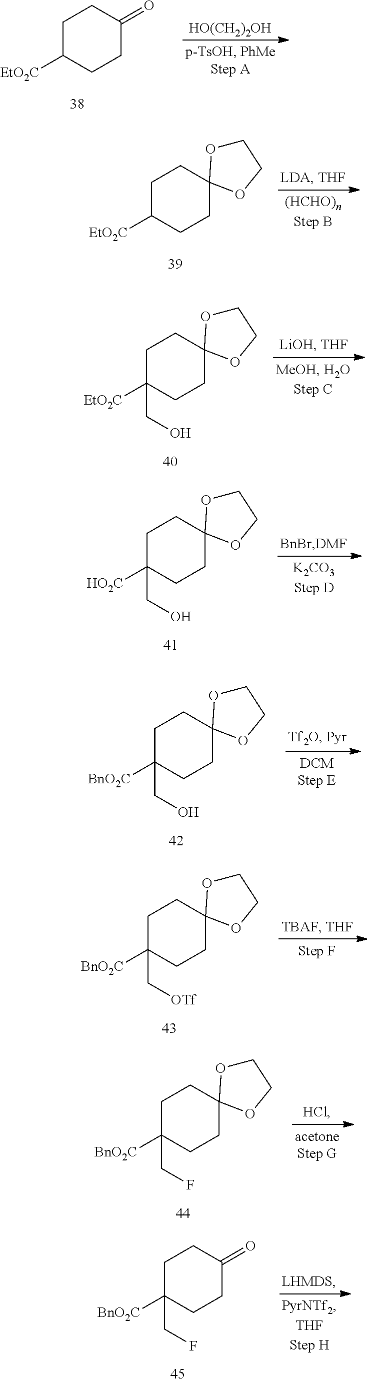

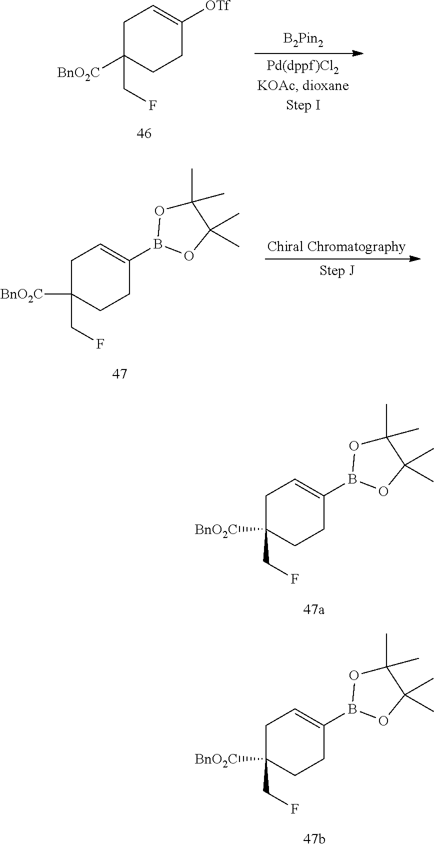

### Step A: Ethyl 1,4-dioxaspiro[4.5]decane-8-carboxylate

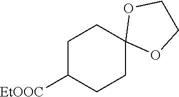 [SMILES: CCOC(=O)C1CCC2(CC1)OCCO2]

A mixture of ethyl 4-oxocyclohexane-1-carboxylate (62 g, 0.36 mol), ethane-1,2-diol (22.3 mL, 0.4 mol) and p-TsOH (0.7 g, 3.6 mmol) in toluene was heated to reflux with a Dean-Stark water trap and a condenser for 24 hr. After cooled down to r.t., the resulting mixture was diluted with EtOAc and washed with sat. NaHCO₃ aq. solution and brine, dried over Na₂SO₄, filtered and concentrated under reduced pressure to give the crude product which was purified by flash chromatography (silica gel, 0-20% EtOAc in PE) to afford the title compound as a colorless oil (70 g, 91% yield). LC-MS (ESI): m/z (M+1)=215.05. ¹H NMR (400 MHz, CDCl₃) b 4.13 (q, J=7.1 Hz, 2H), 3.94 (s, 4H), 2.38-2.28 (m, 1H), 1.99-1.89 (m, 2H), 1.86-1.70 (m, 4H), 1.63-1.50 (m, 2H), 1.25 (t, J=7.1 Hz, 3H).

### Step B: Ethyl 8-(hydroxymethyl)-1,4-dioxaspiro[4.5]decane-8-carboxylate

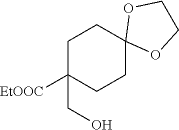 [SMILES: CCOC(=O)C1(CO)CCC2(CC1)OCCO2]

At −78° C., to a solution of ethyl 1,4-dioxaspiro[4.5]decane-8-carboxylate (70 g, 326 mmol) in anhydrous THF (700 mL) was added LDA (2M in THF, 196 mL, 391 mmol) under N₂ atmosphere when maintaining the inner temperature below −60° C. The mixture was stirred at −78° C. for 30 min before the addition of paraformaldehyde (14.8 g, 490 mmol) in one portion. After stirred at r.t. overnight, the resulting mixture was quenched with sat. NH₄Cl aq. solution and extracted with EtOAc. The organic layer was washed with brine, dried over Na₂SO₄, filtered and concentrated under reduced pressure to give the crude product which was purified by flash chromatography (silica gel, 0-40% EtOAc in PE) to afford the title compound as a colorless oil (54 g, 68% yield). LC-MS (ESI): m/z (M+1)=245.23. ¹H NMR (400 MHz, CDCl₃) b 4.20 (q, J=7.1 Hz, 2H), 3.94 (t, J=2.7 Hz, 4H), 3.64 (d, J=6.3 Hz, 2H), 2.19-2.11 (m, J=9.5, 3.8 Hz, 2H), 2.04 (t, J=6.4 Hz, 1H), 1.69-1.52 (m, 6H), 1.28 (t, J=7.1 Hz, 3H).

### Step C: 8-(hydroxymethyl)-1, 4-dioxaspiro[4.5]decane-8-carboxylic acid

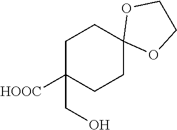 [SMILES: O=C(O)C1(CO)CCC2(CC1)OCCO2]

To a solution of ethyl 8-(hydroxymethyl)-1,4-dioxaspiro[4.5]decane-8-carboxylate (54 g, 221 mmol) in THF (320 mL) and MeOH (105 mL) was added LiOH (3N aqueous solution, 147 mL, 441 mmol). After stirred at 60° C. overnight, the resulting mixture was acidifed with 4N HCl in dioxane to pH 3-4 and concentrated under reduced pressure to give the title compound mixed with LiCl (quant. yield) which was directly used in the next step without purification. LC-MS (ESI): m/z (M+1)=217.30. LC-MS (ESI): m/z (M−1)=215.01. ¹H NMR (400 MHz, MeOD) b 3.90 (s, 4H), 3.48 (s, 2H), 2.09-2.00 (m, 2H), 1.80-1.70 (m, 2H), 1.65-1.55 (m, 2H), 1.53-1.43 (m, 2H).

### Step D: Benzyl 8-(hydroxymethyl)-1,4-dioxaspiro[4.5]decane-8-carboxylate

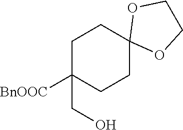

A mixture of 8-(hydroxymethyl)-1,4-dioxaspiro[4.5]decane-8-carboxylic acid (36 g, crude product obtained from the previous step), benzyl bromide (19.7 mL, 166 mmol) and K₂CO₃ (34.5 g, 250 mmol) in DMF (250 mL) was stirred at r.t. overnight. The resulting mixture was filtered and the filtrate was partitioned between EtOAc an H₂O. The layers were separated and the organic layer was washed with brine, dried over Na₂SO₄, filtered and concentrated under reduced pressure to give the crude product which was purified by flash chromatography (silica gel, 0-30% EtOAc in PE) to afford the title compound as a colorless oil (33 g, 65% yield over two steps). LC-MS (ESI): m/z (M+1)=307.51. ¹H NMR (400 MHz, CDCl₃) b 7.42-7.28 (m, 5H), 5.18 (s, 2H), 4.03-3.83 (m, 4H), 3.66 (d, J=6.4 Hz, 2H), 2.20-2.14 (m, 2H), 1.90 (t, J=6.5 Hz, 1H), 1.69-1.58 (m, 6H).

### Step E: Benzyl 8-((((trifluoromethyl)sulfonyl)oxy)methyl)-1,4-dioxaspiro[4.5]decane-8-carboxylate

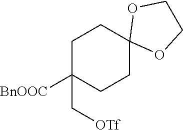

At −10° C., to a solution of benzyl 8-(hydroxymethyl)-1,4-dioxaspiro[4.5]decane-8-carboxylate (33 g, 108 mmol) and pyridine (15.8 mL, 145 mmol) in anhydrous DCM (500 mL) was added Tf₂O (24.5 mL, 145 mmol) dropwise under N₂ atmosphere. After stirred at 0° C. for 2 hr, the resulting mixture was concentrated under reduced pressure and the residue was partitioned between DCM and H₂O. The layers were separated and the organic layer was washed with brine, dried over Na₂SO₄, filtered and concentrated under reduced pressure to give the crude product which was purified by flash chromatography (silica gel, 0-30% EtOAc in PE) to afford the title compound as a yellow oil (40 g, 85% yield). LC-MS (ESI): m/z (M+1)=439.10. ¹H NMR (400 MHz, CDCl₃) b 7.41-7.29 (m, 5H), 5.18 (s, 2H), 4.51 (s, 2H), 3.97-3.87 (m, 4H), 2.28-2.16 (m, 2H), 1.72-1.61 (m, 6H).

### Step F: Benzyl 8-(fluoromethyl)-1,4-dioxaspiro[4.5]decane-8-carboxylate

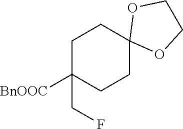

To a solution of benzyl 8-((((trifluoromethyl)sulfonyl)oxy)methyl)-1,4-dioxaspiro[4.5]decane-8-carboxylate (40 g, 91 mmol) in anhydrous THF (300 mL) was added dropwise TBAF (1M in THF, 137 mL, 137 mmol). After stirred at r.t. for 30 min, the resulting mixture was partitioned between EtOAc and H₂O. The layers were separated and the organic layer was washed with brine, dried over Na₂SO₄, filtered and concentrated under reduced pressure to give the crude product which was purified by flash chromatography (silica gel, 0-30% EtOAc in PE) to afford the title compound as a light yellow oil (25 g, 89% yield). LC-MS (ESI): m/z (M+1)=309.10. ¹H NMR (400 MHz, CDCl₃) b 7.41-7.29 (m, 5H), 5.18 (s, 2H), 4.42 (d, J=47.2 Hz, 2H), 3.98-3.87 (m, 4H), 2.26-2.15 (m, 2H), 1.70-1.58 (m, 6H).

### Step G: Benzyl 1-(fluoromethyl)-4-oxocyclohexane-1-carboxylate

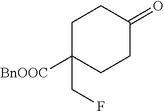

A mixture of benzyl 8-(fluoromethyl)-1,4-dioxaspiro[4.5]decane-8-carboxylate (25 g, 81 mmol) in HCl (1.5N aqueous solution, 216 mL, 324 mmol) and acetone (250 mL) was stirred at r.t. overnight. The resulting mixture was concentrated under reduced pressure and the residue was partitioned between EtOAc and H₂O. The layers were separated and the organic layer was washed with brine, dried over Na₂SO₄, filtered and concentrated under reduced pressure to give the crude product which was purified by flash chromatography (silica gel, 0-30% EtOAc in PE) to afford the title compound as a light yellow oil (20 g, 93% yield). LC-MS (ESI): m/z (M+1)=265.24. ¹H NMR (400 MHz, CDCl₃) b 7.41-7.31 (m, 5H), 5.25 (s, 2H), 4.50 (d, J=47.0 Hz, 2H), 2.50-2.30 (m, 6H), 1.84-1.73 (m, 2H).

### Step H: Benzyl 1-(fluoromethyl)-4-(((trifluoromethyl)sulfonyl)oxy)cyclohex-3-ene-1-carboxylate

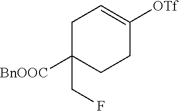

At −78° C., to a solution of benzyl 1-(fluoromethyl)-4-oxocyclohexane-1-carboxylate (20 g, 76 mmol) and 1,1,1-trifluoro-N-(pyridin-2-yl)-N-((trifluoromethyl)sulfonyl)methane sulfonamide (35 g, 98 mmol) in anhydrous THF (200 mL) was added K-HMDS (1M in THF, 113 mL, 113 mmol) dropwise under N₂ atmosphere. After stirred at r.t. overnight, the resulting mixture was quenched with sat. NH₄Cl aq. solution and extracted with EtOAc. The organic layer was washed with brine, dried over Na₂SO₄, filtered and concentrated under reduced pressure to give the crude product which was purified by flash chromatography (silica gel, 0-10% EtOAc in PE) to afford the title compound as a yellow oil (24 g, 86% yield). LC-MS (ESI): m/z (M+1)=397.01. ¹H NMR (400 MHz, CDCl₃) b 7.44-7.27 (m, 5H), 5.75 (t, J=4.0 Hz, 1H), 5.24-5.11 (m, 2H), 4.62-4.37 (m, 2H), 2.83-2.70 (m, 1H), 2.45-2.15 (m, 4H), 1.98-1.85 (m, 1H).

### Step I: Benzyl 1-(fluoromethyl)-4-(4,4,5,5-tetramethyl-1,3,2-dioxaborolan-2-yl)cyclo hex-3-ene-1-carboxylate

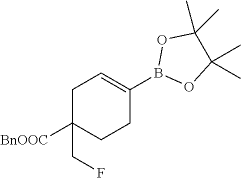

A mixture of benzyl 1-(fluoromethyl)-4-(((trifluoromethyl)sulfonyl)oxy)cyclohex-3-ene-1-carboxylate (24 g, 61 mmol), 4,4,4′,4′,5,5,5′,5′-octamethyl-2,2′-bi(1,3,2-dioxaboro lane) (17 g, 68 mmol), Pd(dppf)Cl₂ (5.3 g, 6.5 mmol) and KOAc (19 g, 195 mmol) in dioxane (200 mL) was stirred at 80° C. under N₂ atmosphere overnight. The resulting mixture was filtered through a pad of Celite and the filtrate was partitioned between EtOAc and H₂O. The layers were separated and the organic layer was washed with brine, dried over Na₂SO₄, filtered and concentrated under reduced pressure to give the crude product which was purified by flash chromatography (silica gel, 0-15% EtOAc in PE) to afford the title compound as a colorless oil (18 g, 79% yield). LC-MS (ESI): m/z (M+1)=375.40. ¹H NMR (400 MHz, CDCl₃) b 7.41-7.26 (m, 5H), 6.51 (s, 1H), 5.16 (s, 2H), 4.63-4.37 (m, 2H), 2.70-2.58 (m, 1H), 2.23-2.10 (m, 3H), 1.95-1.75 (m, 2H), 1.25 (s, 12H).

### Step J: Benzyl (R)-1-(fluoromethyl)-4-(4,4,5,5-tetramethyl-1,3,2-dioxaborolan-2-yl)cyclohex-3-ene-1-carboxylate and benzyl (S)-1-(fluoromethyl)-4-(4,4,5,5-tetramethyl-1,3,2-dioxaborolan-2-yl)cyclohex-3-ene-1-carboxylate

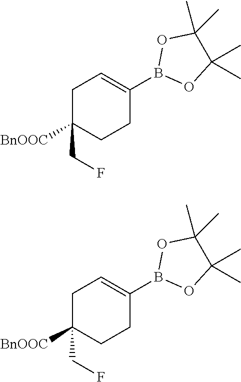

Benzyl 1-(fluoromethyl)-4-(4,4,5,5-tetramethyl-1,3,2-dioxaborolan-2-yl)cyclohex-3-ene-1-carboxylate (9.4 g, 25 mmol) was separated by chiral chromatography by using the condition (Column: Regis (S,S)-whelk-O1; Column size: 0.46 cm I.D.×25 cm L; Injection: 5 ul; Mobile phase: CO₂/[hexane:IPA=4:1, (v/v)]=80/20; Flow rate: 2.0 ml/min; Wave length: UV 220 nm; Temperature: 35° C.) to give benzyl (R)-1-(fluoro methyl)-4-(4,4,5,5-tetramethyl-1,3,2-dioxaborolan-2-yl)cyclohex-3-ene-1-carboxylate (second eluent, Peak B, 2.8 g, 7.5 mmol) and benzyl (S)-1-(fluoromethyl)-4-(4,4,5,5-tetramethyl-1,3,2-dioxaborolan-2-yl)cyclohex-3-ene-1-carboxylate (first eluent, Peak A, 2.9 g, 7.7 mmol). The isomers have been tentatively assigned to the indicated absolute stereochemistry. Compounds described below using these assignments are dependent on this tentative assignment.

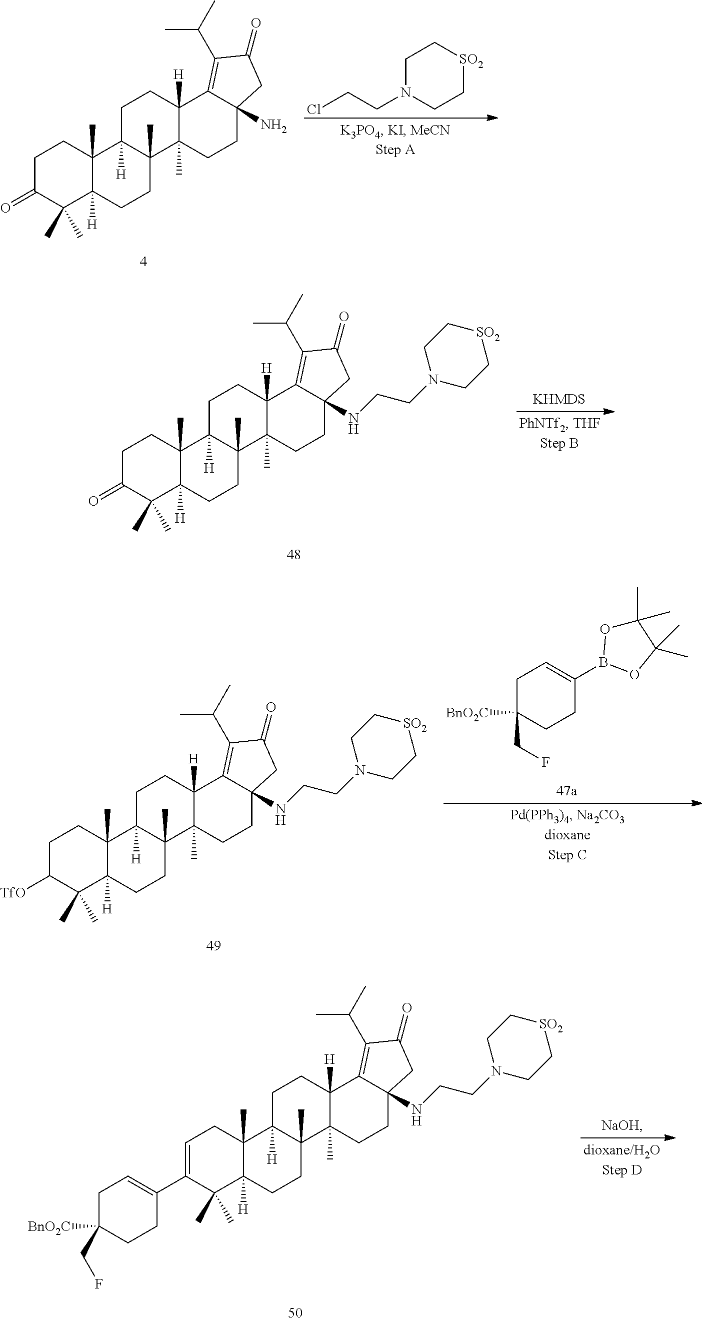

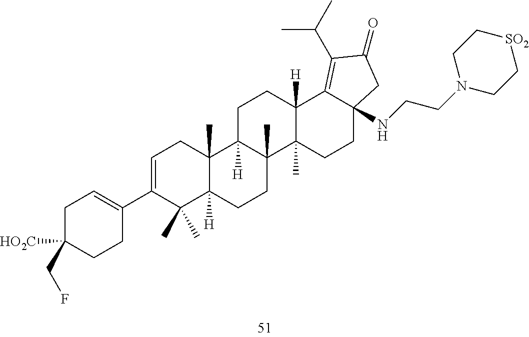

### Step A: (3aR,5aR,5bR,7aR,11aR,11bR,13aS)-3a-((2-(1,1-dioxidothiomorpholino)ethyl) amino)-1-isopropyl-5a,5b,8,8,11a-pentamethyl-3,3a,4,5,5a,6,7,7a,8,10,11,11a,11b,12,13,13a-hexadecahydro-2H-cyclopenta[a]chrysene-2,9(5bH)-dione

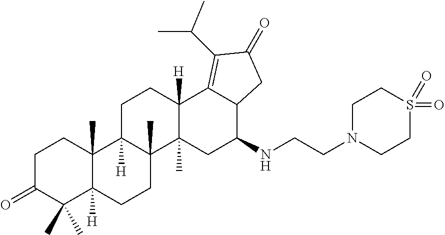

A mixture of (3aR,5aR,5bR,7aR,11aR,11bR,13aS)-3a-amino-1-isopropyl-5a,5b,8,8,11a-pentamethyl-3,3a,4,5,5a,6,7,7a,8,10,11,111a,11 b,12,13,13a-hexadecahydro-2H-cyclopenta[a]chrysene-2,9(5bH)-dione (400 mg, 0.9 mmol), 4-(2-chloroethyl)thiomorpholine 1,1-dioxide (198 mg, 1.0 mmol), K₃PO₄ (579 mg, 2.7 mmol) and KI (226 mg, 1.4 mmol) in MeCN (4 mL) was stirred at 100° C. under N₂ atmosphere overnight. The resulting mixture was diluted with EtOAc and filtered. The filtrate was partitioned between EtOAc and H₂O and the organic layer was washed with brine, dried over Na₂SO₄, filtered and concentrated under reduced pressure to give the crude product which was purified by flash chromatography (silica gel, 0-10% MeOH in DCM) to afford the title compound as a light yellow solid (300 mg, 56% yield). LC-MS (ESI): m/z (M+1)=601.39. ¹H NMR (400 MHz, CDCl₃) b 3.17-3.12 (m, 1H), 3.11-2.81 (m, J=17.4 Hz, 12H), 2.69-2.59 (m, 2H), 2.58-2.38 (m, 4H), 2.37-2.24 (m, 2H), 2.13-1.85 (m, 6H), 1.54-1.37 (m, 8H), 1.27-1.16 (m, 9H), 1.11-0.92 (m, 12H).

### Step B: (3aR,5aR,5bR,7aR,11aR,11bR,13aS)-3a-((2-(1,1-dioxidothiomorpholino)ethyl) amino)-1-isopropyl-5a,5b,8,8,11a-pentamethyl-2-oxo-3,3a,4,5,5a,5b,6,7,7a,8,11,11a,11b,12,13,13a-hexadecahydro-2H-cyclopenta[a]chrysen-9-yltrifluoromethanesulfonate

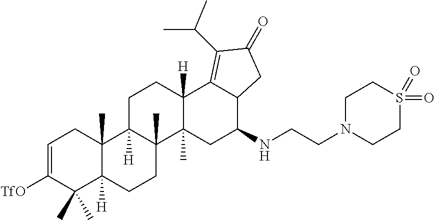

At −78° C., to a solution of (3aR,5aR,5bR,7aR,11 aR,11 bR,13aS)-3a-((2-(1,1-dioxidothio morpholino)ethyl)amino)-1-isopropyl-5a,5b,8,8,11a-pentamethyl-3,3a,4,5,5a,6,7,7a,8,10,11,111a,11 b,12,13,13a-hexadecahydro-2H-cyclopenta[a]chrysene-2,9(5bH)-dione (300 mg, 0.5 mmol) and 1,1,1-trifluoro-N-phenyl-N-((trifluoromethyl)sulfonyl)methane sulfonamide (267 mg, 0.75 mmol) in anhydrous THF (5 mL) was added K-HMDS (1M in THF, 1 mL, 1 mmol) dropwise under N₂ atmosphere. After stirred at r.t. overnight, the resulting mixture was quenched with sat. NH₄Cl aq. solution and extracted with DCM. The organic layer was washed with brine, dried over Na₂SO₄, filtered and concentrated under reduced pressure to give the crude product which was purified by flash chromatography (silica gel, 0-10% MeOH in DCM) to afford the title compound as a light yellow solid (130 mg, 36% yield). LC-MS (ESI): m/z (M+1)=733.26.

### Step C: Benzyl (R)-4-((3aR,5aR,5bR,7aR,11aS,11bR,13aS)-3a-((2-(1,1-dioxidothio morpholino)ethyl)amino)-1-isopropyl-5a,5b,8,8,11a-pentamethyl-2-oxo-3,3a,4,5,5a,5b,6,7,7a,8,11,11a,11b,12,13,13a-hexadecahydro-2H-cyclopenta[a]chrysen-9-yl)-1-(fluoromethyl)cyclohex-3-ene-1-carboxylate

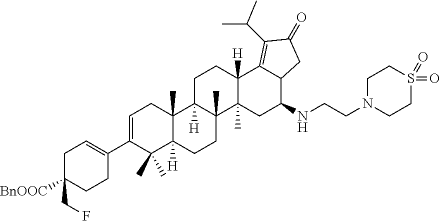

A mixture of (3aR,5aR,5bR,7aR,1 aR,11bR,13aS)-3a-((2-(1,1-dioxidothiomorpholino)ethyl)amino)-1-isopropyl-5a,5b,8,8,11a-pentamethyl-2-oxo-3,3a,4,5,5a,5b,6,7,7a,8,11,111a,11 b,12,13,13a-hexadecahydro-2H-cyclopenta[a]chrysen-9-yltrifluoromethanesulfonate (60 mg, 0.08 mmol), benzyl (R)-1-(fluoromethyl)-4-(4,4,5,5-tetramethyl-1,3,2-dioxaborolan-2-yl)cyclohex-3-ene-1-carboxylate (37 mg, 0.1 mmol), Pd(PPh₃)₄ (20 mg, 0.018 mmol) and Na₂CO₃ (28 mg, 0.27 mmol) in dioxane (1.5 mL) and H₂O (0.5 mL) was stirred at 80° C. under N₂ atmosphere for 3 hr. The resulting mixture was partitioned between DCM and H₂O and the organic layer was washed with brine, dried over Na₂SO₄, filtered and concentrated under reduced pressure to give the crude product which was purified by flash chromatography (silica gel, 0-10% MeOH in DCM) to afford the title compound as a light yellow solid (40 mg, 60% yield). LC-MS (ESI): m/z (M+1)=831.52.

### Step D: (R)-4-((3aR,5aR,5bR,7aR,11aS,11bR,13aS)-3a-((2-(1,1-dioxidothiomorpholino)ethyl)amino)-1-isopropyl-5a,5b,8,8,11a-pentamethyl-2-oxo-3,3a,4,5,5a,5b,6,7,7a,8,11,11a,11b,12,13,13a-hexadecahydro-2H-cyclopenta[a]chrysen-9-yl)-1-(fluoromethyl)cyclohex-3-ene-1-carboxylic acid

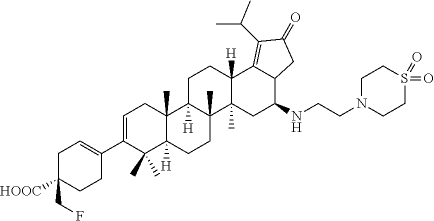

A mixture of benzyl (R)-4-((3aR,5aR,5bR,7aR,11aS,11 bR,13aS)-3a-((2-(1,1-dioxido thiomorpholino)ethyl)amino)-1-isopropyl-5a,5b,8,8,11a-pentamethyl-2-oxo-3,3a,4,5,5a,5b,6,7,7a,8,11,111a,11 b,12,13,13a-hexadecahydro-2H-cyclopenta[a]chrysen-9-yl)-1-(fluoromethyl)cyclohex-3-ene-1-carboxylate (40 mg, 0.048 mmol) in 1N NaOH (0.5 mL), MeOH (0.1 mL) and dioxane (0.5 mL) was stirred at 80° C. for 2 hr. The resulting mixture was acidified with 1N HCl to pH 3-4 and extracted with DCM. The organic layer was washed with brine, dried over Na₂SO₄, filtered and concentrated under reduced pressure to give the crude product which was purified by HPLC (C18, 0-100% MeCN in H₂O with 0.1% formic acid). A few drops of 4N HCl in dioxane was added to the fraction collected by HPLC to afford the title compound as HCl salt (8 mg, 22% yield). LC-MS (ESI): m/z (M+1)=741.40. ¹H NMR (400 MHz, MeOD) b 5.35 (s, 1H), 5.23 (d, J=4.6 Hz, 1H), 4.54 (dd, J=18.8, 8.8 Hz, 1H), 4.42 (dd, J=18.6, 8.8 Hz, 1H), 3.30-3.21 (m, 8H), 3.13-1.34 (m, 30H), 1.30-0.91 (m, 21H).

### Example 11 (Compound 53)

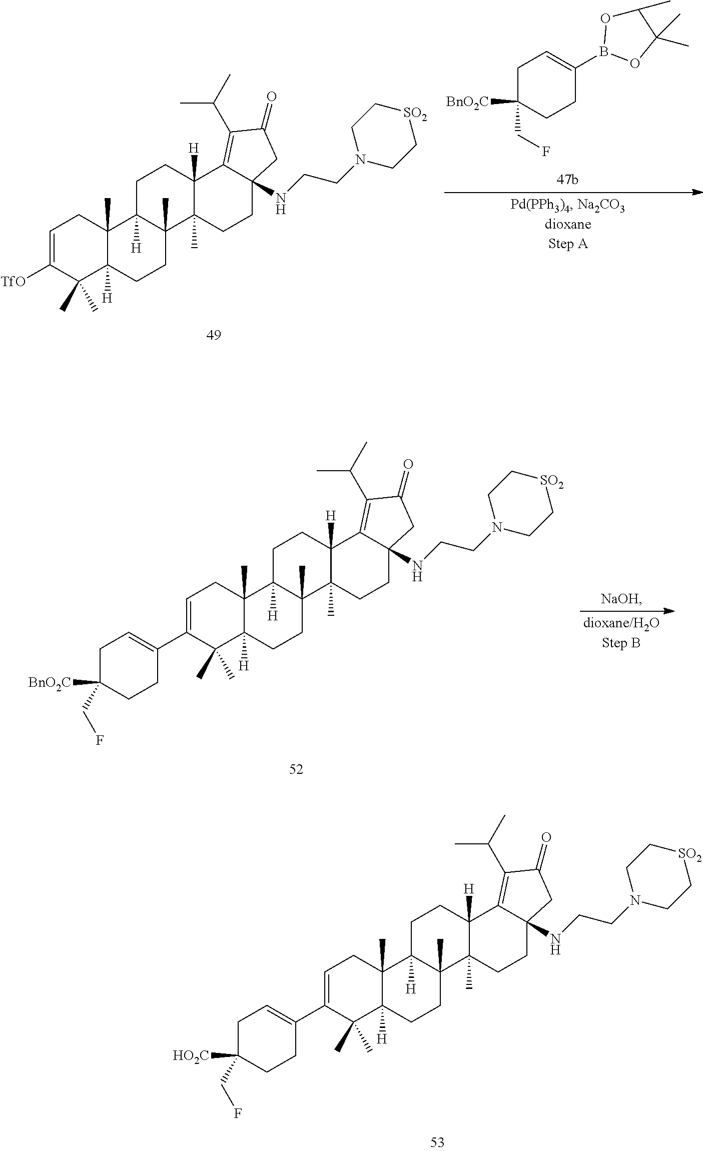

### Step A: Benzyl (S)-4-((3aR,5aR,5bR,7aR,11aS,11bR,13aS)-3a-((2-(1,1-dioxidothio morpholino)ethyl)amino)-1-isopropyl-5a,5b,8,8,11a-pentamethyl-2-oxo-3,3a,4,5,5a,5b,6,7,7a,8,11,11a,11b,12,13,13a-hexadecahydro-2H-cyclopenta[a]chrysen-9-yl)-1-(fluoromethyl)cyclohex-3-ene-1-carboxylate

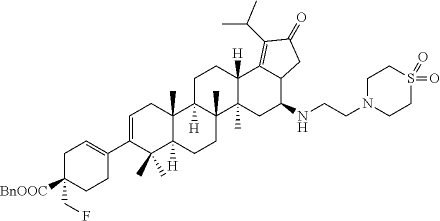

A mixture of (3aR,5aR,5bR,7aR,1 aR,11 bR,13aS)-3a-((2-(1,1-dioxidothiomorpholino)ethyl)amino)-1-isopropyl-5a,5b,8,8,11a-pentamethyl-2-oxo-3,3a,4,5,5a,5b,6,7,7a,8,11,111a,11 b,12,13,13a-hexadecahydro-2H-cyclopenta[a]chrysen-9-yltrifluoromethanesulfonate (60 mg, 0.08 mmol), benzyl (S)-1-(fluoromethyl)-4-(4,4,5,5-tetramethyl-1,3,2-dioxaborolan-2-yl)cyclohex-3-ene-1-carboxylate (37 mg, 0.1 mmol), Pd(PPh₃)₄ (20 mg, 0.018 mmol) and Na₂CO₃ (28 mg, 0.27 mmol) in dioxane (1.5 mL) and H₂O (0.5 mL) was stirred at 80° C. under N₂ atmosphere for 2 hr. The resulting mixture was partitioned was partitioned between DCM and H₂O and the organic layer was washed with brine, dried over Na₂SO₄, filtered and concentrated under reduced pressure to give the crude product which was purified by flash chromatography (silica gel, 0-10% MeOH in DCM) to afford the title compound as a light yellow solid (45 mg, 68% yield). LC-MS (ESI): m/z (M+1)=831.53.

### Step B: (S)-4-((3aR,5aR,5bR,7aR,11aS,11bR,13aS)-3a-((2-(1,1-dioxidothiomorpholino)ethyl)amino)-1-isopropyl-5a,5b,8,8,11a-pentamethyl-2-oxo-3,3a, 4,5,5a,5b,6,7,7a,8,11,11a,11b,12,13,13a-hexadecahydro-2H-cyclopenta[a]chrysen-9-yl)-1-(fluoromethyl)cyclohex-3-ene-1-carboxylic acid

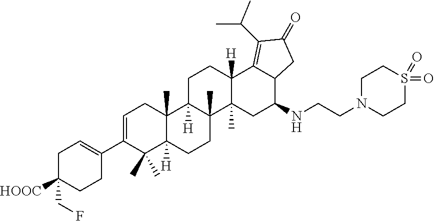

A mixture of benzyl (S)-4-((3aR,5aR,5bR,7aR,11aS,11 bR,13aS)-3a-((2-(1,1-dioxido thiomorpholino)ethyl)amino)-1-isopropyl-5a,5b,8,8,11a-pentamethyl-2-oxo-3,3a,4,5,5a,5b,6,7,7a,8,11,111a,11 b,12,13,13a-hexadecahydro-2H-cyclopenta[a]chrysen-9-yl)-1-(fluoromethyl)cyclohex-3-ene-1-carboxylate (45 mg, 0.054 mmol) in 1N NaOH (0.5 mL), MeOH (0.1 mL) and dioxane (0.5 mL) was stirred at 80° C. for 2 hr. The resulting mixture was acidified with 1N HCl to pH 3-4 and extracted with DCM. The organic layer was washed with brine, dried over Na₂SO₄, filtered and concentrated under reduced pressure to give the crude product which was purified by HPLC (C18, 0-100% MeCN in H₂O with 0.1% formic acid). A few drops of 4N HCl in dioxane was added to the fraction collected by HPLC to afford the title compound as HCl salt (13 mg, 32% yield). LC-MS (ESI): m/z (M+1)=741.52. ¹H NMR (400 MHz, MeOD) b 5.34 (s, 1H), 5.23 (d, J=4.7 Hz, 1H), 4.55 (dd, J=18.5, 8.8 Hz, 1H), 4.43 (dd, J=18.4, 8.8 Hz, 1H), 3.30-3.14 (m, 8H), 3.07-1.41 (m, 30H), 1.31-1.17 (m, 9H), 1.15-0.81 (m, 12H).

### MT4 Cell Antiviral Assay

### Experimental Procedure

Antiviral HIV activity and compound-induced cytotoxicity were measured in parallel by means of a propidium iodide based procedure in the human T-cell lymphotropic virus transformed cell line MT4. Aliquots of the test compounds were serially diluted in medium (RPMI 1640, 10% fetal calf serum (FCS), and gentamycin) in 96-well plates (Costar 3598) using a Cetus Pro/Pette. Exponentially growing MT4 cells were harvested and centrifuged at 1000 rpm for 10 min in a Jouan centrifuge (model CR 4 12). Cell pellets were resuspended in fresh medium (RPMI 1640, 20% FCS, 20% IL-2, and gentamycin) to a density of 5×105 cells/ml. Cell aliquots were infected by the addition of HIV-1 (strain IIIB) diluted to give a viral multiplicity of infection of 100×TCID50. A similar cell aliquot was diluted with medium to provide a mock-infected control. Cell infection was allowed to proceed for 1 hr at 37° C. in a tissue culture incubator with humidified 5% CO₂ atmosphere. After the 1 hr incubation the virus/cell suspensions were diluted 6-fold with fresh medium, and 125 μl of the cell suspension was added to each well of the plate containing pre-diluted compound. Plates were then placed in a tissue culture incubator with humidified 5% CO₂ for 5 days. At the end of the incubation period, cell number and hence HIV-induced cytopathy was estimated by either (A) propidium iodide staining, or by an (B) MTS tetrazolium staining method.

For propidium iodide readout, 27 μl of 5% Nonidet-40 was added to each well of the incubation plate. After thorough mixing with a Costar multitip pipetter, 60 μl of the mixture was transferred to filter-bottomed 96-well plates. The plates were analyzed in an automated assay instrument (Screen Machine, Idexx Laboratories). The control and standard used was 3′-azido-3′-deoxythymidine tested over a concentration range of 0.01 to 1 μM in every assay. The expected range of IC₅₀ values for 3′-azido-3′-deoxythymidine is 0.04 to 0.12 μM. The assay makes use of a propidium iodide dye to estimate the DNA content of each well.

For MTS readout, 20 μl CellTiter 96 AQ One Solution reagent (Promega #G3582) was added to each well. At 75 minutes following the addition of MTS reagent, absobance was read at 492 nM using a Tecan Sunrise 96-well plate reader.

### Analysis:

The antiviral effect of a test compound is reported as an EC₅₀, i.e. the inhibitory concentration that would produce a 50% decrease in the HIV-induced cytopathic effect. This effect is measured by the amount of test compound required to restore 50% of the cell growth of HIV-infected MT4 cells, compared to uninfected MT4 cell controls. ICso was calculated by RoboSage, Automated Curve Fitting Program, version 5.00, 10 Jul. 1995.

For each assay plate, the results (relative fluorescence units, rfU, or OD values) of wells containing uninfected cells or infected cells with no compound were averaged, respectively. For measurements of compound-induced cytotoxicty, results from wells containing various compound concentrations and uninfected cells were compared to the average of uninfected cells without compound treatment. Percent of cells remaining is determined by the following formula:

Percent of cells remaining=(compound-treated uninfected cells,rfU, or OD values/untreated uninfected cells)×100.

A level of percent of cells remaining of 79% or less indicates a significant level of direct compound-induced cytotoxicity for the compound at that concentration.

When this condition occurs the results from the compound-treated infected wells at this concentration are not included in the calculation of EC₅₀.

For measurements of compound antiviral activity, results from wells containing various compound concentrations and infected cells are compared to the average of uninfected and infected cells without compound treatment. Percent inhibition of virus is determined by the following formula:

Percent inhibition of virus=(1−((ave. untreated uninfected cells−treated infected cells)/(ave. untreated uninfected cells−ave. untreated infected cells)))×100.

The following table summarizes EC₅₀ values for the compounds of the above Examples.

| Example | EC₅₀ NL4-3 wt | EC₅₀ V370A |
|---|---|---|
| number | (nM) | (nM) |
| 1 | 99 | 352 |
| 2 | 8 | 18 |
| 3 | 11 | 42 |
| 4 | 29 | ND |
| 5 | 30 | 776 |
| 6 | 159 | ND |
| 7 | 6.3 | ND |
| 8 | 2 | 8 |
| 9 | ND | ND |
| 10 | ND | ND |
| 11 | ND | ND |

1. A compound of Formula I: 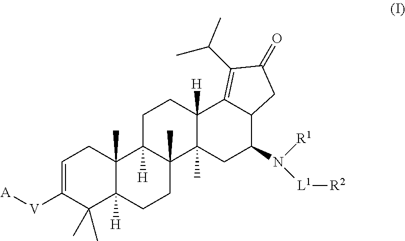
or a pharmaceutically acceptable salt thereof, wherein:
R¹ is H or C₁₋₄alkyl;
L¹ is a bond, C₁₋₈alkylene, or C₀₋₃alkyleneC(O)C₀₋₃alkylene;
R² is N(R³)R⁴, or —OR³, wherein each R³ and R⁴ are independently H, C₁₋₃alkyl, C₁₋₃alkylene-C₃₋₆cycloalkyl, C₁₋₃alkylene-C₅₋₇heterocycle, C₁₋₃alkyleneN(C₁₋₃alkyl)₂, C₁₋₃alkyleneC₅₋₇aryl, C(O)C₅₋₇aryl, C(O)C₁₋₄alkyl, C₁₋₃alkyleneC₅₋₇heteroaryl wherein each R³ and R⁴ may be substituted with one or two substituents independently selected from halogen, C₁₋₄alkyl, SO₂C₁₋₃alkyl; or
when R² is N(R³)R⁴, R³ and R⁴ may bond together and along with the N to which they are bonded form a 4 to 7-membered heterocycle, and said heterocycle may contain one other hetero atom selected from N, S, and O and wherein said S atom may be substituted by up to 2 oxo groups, and wherein said heterocycle may be substituted with one or two substituents independently selected from halogen, C₁₋₄alkyl, SO₂C₁₋₃alkyl;
V is C₄₋₈cycloalkylene, C₄₋₈cycloalkenylene, C₅₋₈aryl, or 5 to 8-membered heteroaryl ring, wherein V may be substituted with one or two substituents independently selected from halogen and C₁₋₃alkyl wherein said alkyl may be substituted with one or 2 halogens; and
A is —CO₂C₁₋₃alkyl or CO₂H.

2. A compound or salt according to claim 1 wherein R¹ is H.

3. A compound or salt according to claim 1 wherein L¹ is C₁₋₄alkylene, C(O), or C(O)C₁₋₄alkylene.

4. A compound or salt according to claim 1 wherein R² is N(R³)(R⁴).

5. A compound or salt according to claim 1 wherein R³ and R⁴ are independently C₁₋₃alkyleneN(C₁₋₃alkyl)₂, or C₁₋₃alkylenephenyl, wherein each R³ and R⁴ may be substituted with one substituent independently selected from halogen or SO₂C₁₋₃alkyl; or R³ and R⁴ are bonded together and along with the N to which they are bonded form a 6-membered heterocycle, and said heterocycle may contain one S atom and wherein said S atom may be substituted by 2 oxo groups; and wherein each R³ and R⁴ may be substituted with one or two substituents independently selected from halogen, C₁₋₄alkyl, SO₂C₁₋₃alkyl.

6. A compound or salt according to claim 1 wherein V is phenylene or cyclohexenylene optionally substituted with halogen or C₁₋₃alkyl wherein said alkyl may be substituted with one or 2 halogens.

7. A compound or salt according to claim 1 wherein A is —CO₂H.

8. A salt according to claim 1 wherein the salt is a base salt.

9. A salt according to claim 8 wherein the salt is a Lysine salt.

10. A pharmaceutical composition comprising a compound or salt according to claim 1.

11. A method of treating an HIV infection in a human comprising administering to the subject a pharmaceutical composition according to claim 10.

12. A method of preventing an HIV infection in a subject at risk for developing an HIV infection, comprising administering to the subject pharmaceutical composition according to claim 10.

13-14. (canceled)
